(19) United States
(12) Patent Application Publication
(10) Pub. No.: US 2024/0115831 A1
(43) Pub. Date: Apr. 11, 2024

(54) ENHANCED MEDITATION EXPERIENCE BASED ON BIO-FEEDBACK

(71) Applicant: Apple Inc., Cupertino, CA (US)

(72) Inventors: Christine E. Welch, San Jose, CA (US); Alastair K. Fettes, San Francisco, CA (US); Amy E. DeDonato, San Francisco, CA (US); Christine Godwin, Sunnyvale, CA (US); Dorian D. Dargan, Oakland, CA (US); Eric Landreneau, Castro Valley, CA (US); Gary I. Butcher, Los Gatos, CA (US); Grant H. Mulliken, Los Gatos, CA (US); Hugh A. Sider, San Jose, CA (US); Izzet B. Yildiz, Sunnyvale, CA (US); James D. Dusseau, Santa Clara, CA (US); Jean-Francois St-Amour, Mountain View, CA (US); Joanne Lee, Sunnyvale, CA (US); Lucie Belanger, Beloeil, Quebec (CA); Michael B. Tucker, Marina Del Rey, CA (US); Philipp Rockel, San Francisco, CA (US); Theodore Nestor Panagiotopoulos, San Jose, CA (US)

(21) Appl. No.: 18/390,248

(22) Filed: Dec. 20, 2023

Related U.S. Application Data
(63) Continuation of application No. PCT/US2022/036068, filed on Jul. 5, 2022.
(60) Provisional application No. 63/218,617, filed on Jul. 6, 2021.
(60) Provisional application No. 63/350,170, filed on Jun. 8, 2022.

(51) Int. Cl. A61M 21/02; G06F 3/01; G16H 20/70; G16H 40/63

(52) U.S. Cl. CPC A61M 21/02; G06F 3/015; G16H 20/70; G16H 40/63; A61M 2021/0027

(57) ABSTRACT

Various implementations disclosed herein include devices, systems, and methods that provide customized feedback content during a meditation experience. For example, an example process may include obtaining physiological data via one or more sensors, determining that an attentive state based on the physiological data, customizing feedback content based on a user attribute to change the attentive state during the meditation mode, and providing the customized feedback content during the meditation mode after a delay time based on the user attribute.

### CROSS-REFERENCE TO RELATED APPLICATIONS

This patent application is a continuation of International Application No. PCT/US2022/036068 filed Jul. 5, 2022, which claims priority to U.S. Provisional Application No. 63/218,617 filed Jul. 6, 2021, and U.S. Provisional Application No. 63/350,170 filed Jun. 8, 2022, entitled “ENHANCED MEDITATION EXPERIENCE BASED ON BIO-FEEDBACK,” each of which is incorporated herein by this reference in its entirety.

### TECHNICAL FIELD

The present disclosure generally relates to presenting content via electronic devices, and in particular, to systems, methods, and devices that determine a respiratory state and attention state during and/or based on the presentation of electronic content.

### BACKGROUND

Electronic devices may be used to help users engage in various experiences in which particular user states are desired. For example, an electronic device may be used to present content that guides a user through a meditation experience in which the user desires to relax and/or focus on something such as his or her breathing. Such content is generally not responsive or adaptive to the user's actual state and thus may not be as effective or efficient as desired.

### SUMMARY

Various implementations disclosed herein include devices, systems, and methods that present content and assess a respiratory state (e.g., 7 breaths per minute, etc.) and an attention state (e.g., whether focused or mind wandering) based on physiological data (e.g., heart rate, respiration rate, body temperature, electrocardiogram, blood oxygen saturation, skin conductance, and the like) and modify (e.g., enhance) the content based on the respiratory state and attention state. For example, a visual and/or audio notification may be provided to the user to focus. Additionally, respiratory and attention statistical analysis and summary information may be provided.

A respiratory state and attention state while viewing and/or listening to content on an electronic device can have a significant effect on the experience. For example, staying focused and engaged may be required for meaningful experiences, such as meditation, watching educational or entertaining content, learning a new skill, and the like. Improved techniques for assessing the respiratory and attention states of users viewing and interacting with content may enhance a user's enjoyment, comprehension, and learning of the content. Moreover, content may not be presented in a way that makes sense to a particular user. Content creators and systems may be able to provide better and more tailored user experiences that a user is more likely to enjoy, comprehend, and learn from based on respiratory state and attention state information (e.g., a more meaningful meditation experience).

In an exemplary implementation, devices, systems, and methods facilitate meditation based on tracking a user's respiration and attention state using sensors that obtain physiological data. The meditation may (but does not necessarily) guide the user to be attentive to his or her breathing. The system may identify a baseline user state and a goal user state and enhance the experience to achieve the goal. In addition to respiration and attention, sensor data may be used to determine electroencephalography (EEG), temperature (e.g., on nose), heartrate (e.g., on forehead), and the like. In some implementations, the content enhancement may be selected based on a characteristic of an environment of the user (e.g., real-world physical environment, a virtual environment, or a combination of each). The device (e.g., a handheld, laptop, desktop, or head-mounted device (HMD)) provides content (e.g., a visual and/or auditory experience) corresponding to the real-world physical environment, a virtual reality environment, or a combination of such environments to the user. The content (e.g., an extended reality (XR) environment) may be enhanced visual/audio content and guidance that provides a closed loop meditation experience based on real time bio-feedback.

In some implementations, the device obtains, with one or more sensors, physiological data (e.g., respiratory data, image data (facial, body, etc.), EEG amplitude, pupil modulation, eye gaze saccades, etc.) associated with the user. Based on the obtained physiological data, some of the techniques described herein determine a user's respiratory state and attention state (e.g., attentive, mind-wandering, etc.) during the experience (e.g., a meditation experience). For example, some implementations may identify that the user's eye characteristic (e.g., blink rate, stable gaze direction, saccade amplitude/velocity, and/or pupil radius) correspond to a “focused” attention state rather than a “mind wandering” attention state. Based on the physiological data and associated physiological response, the techniques can provide feedback to the user that the current respiratory and/or attention state differs from an intended state of the experience, recommend similar content or similar portions of the experience, and/or adjust, enhance, or otherwise modify the content.

In some implementations, a content modification may be the start or end of a meditation experience or a change during an ongoing experience. Some implementations may provide a visualization of a current respiratory state or a desired/improved respiratory state. For example, respiratory oscillations may be visualized by the user by displaying a shrinking and enlarging flower (e.g., for each breath), or a virtual candle being blown out by the user's breath. Some implementations may use subtle cues to change from lighter to darker ambience and/or bells, audio, or chimes. Some implementations may escalate explicitness from subtle cues (e.g., small icons that blend with the background) to direct instructions (e.g., “control your breathing”). Some implementations may use spatialized audio to redirect attention (e.g., a bird chirping at a particular location of the content). Some implementations may provide simultaneous/combined feedback regarding the current and desired respiratory state and/or attention state.

Some implementations improve respiratory and attention state assessment accuracy, e.g., improving the assessment of a user's attention to a task (e.g., notifying the user they are mind wandering and/or not breathing appropriately during a meditation experience). Some implementations improve user experiences by providing attention/respiration assessments that minimize or avoid interrupting or disturbing user experiences, for example, without significantly interrupting a user's attention or ability to perform a task.

In some implementations, the respiratory rate (e.g., breath tracking) may involve sensor fusion of two or more different sensor data. For example, head pose from an inertial measurement unit (IMU), audio from a microphone, images from one or more cameras (e.g., a jaw cam, a down cam of the body, an eye cam for tissue around the eye, and the like), motion of the body, and/or signal of the face modulated by the breath (e.g., remote photoplethysmogram (PPG)). Using this type of sensor fusion to track the breathing of the user, such as while wearing an HMD, may negate the need for a user to wear a sensor worn around the user's diaphragm, for example.

Physiological response data, such as EEG amplitude/frequency, pupil modulation, eye gaze saccades, etc., can depend on the attention state of an individual and characteristics of the scene in front of him or her and the content enhancement that is presented therein. Physiological response data can be obtained while using a device with eye tracking technology while users perform tasks that demand varying levels of attention, such as focused attention to a meditation or educational video (e.g., an instructional cooking video). In some implementations, physiological response data can be obtained using other sensors, such as EEG sensors. Observing repeated measures of physiological response data to an experience can give insights about the underlying respiratory and attention state of the user at different time scales. These metrics of respiration and attention can be used to provide feedback during a learning experience.

Some implementations use scene analysis that identifies relevant areas of the content (e.g., creating an attention map based on object detection, facial recognition, etc.) to determine what the person is looking at during the presentation of content. Such scene analysis and/or determination of a looked upon object may be used to improve the determination of the user's respiratory and attention states.

In some implementations, meditation may be recommended (e.g., at a particular time, place, task, etc.) based on the user's respiratory state and attention state (e.g., focused, distracted, etc.) by identifying a type or characteristic of the recommended meditation based on various factors (e.g., physical environment context, scene understanding of what the user is seeing in an XR environment, and the like). For example, one type of meditation may be recommended in one circumstance (e.g., mindfulness meditation for mind wandering) and a different type of meditation may be recommended in another circumstance (e.g., movement/physical meditation for distracted or anxiety situations). If the user wants a focused-attention session (e.g., focused on a single task like watching a video) and if it is detected that the user is distracted, an open-monitoring meditation can be recommended. For example, open monitoring meditation can allow and/or encourage the user to notice multiple sounds/visuals/thoughts in the environment of the meditation, and could replenish his or her ability to focus on a single item. Additionally, or alternatively, if the user wants to multi-task using various applications, and the system detects that the user is overwhelmed, the system could suggest that he or she perform focus-attention mediation techniques (e.g., attention to breath by focusing on a breathing monitor icon, such as a flower that moves with each breath or blowing out a candle). The focus-attention mediation techniques could allow the user to regain the ability to focus on a single item at a time. In an exemplary implementation, a meditation session could be initiated for the user which may be in opposition of the main task he or she is aiming to accomplish such that he or she can relax/replenish during meditation and return to task at hand more effectively.

Experiences other than meditation experiences can utilize the techniques described herein regarding assessing respiratory states and attention states. For example, an education experience could notify a pupil to focus on an education task when he or she appears to be mind wandering. Another example may be a workplace experience of notifying a worker who needs to be focused on his or her current task. For example, providing feedback to a surgeon who may be experiencing minor fatigue during a long surgery, alerting a truck driver on a long drive that he or she is losing focus and may need to pull over to sleep, and the like. The techniques described herein can be customized to any user and experience that may need some type of content enhancement to enter or maintain one or more particular respiratory states and attention states.

The present technology may gather and use data from various sources to provide feedback content during a meditation. This data, in some instances, may include personal information data that uniquely identifies or may be used to locate or contact a specific individual. This personal information data may include location-based data, demographic data, telephone numbers, email addresses, social media account names, home or work addresses, data or records associated with a user's health or fitness level (e.g., information associated with vital signs, medication, exercise, and the like), date of birth, or other personal or identifying information.

It is recognized that, in some instances, such personal information data may be used to benefit users. For example, the personal information data may be used to provide feedback content during a meditation. Accordingly, use of such personal information data enables calculated control of the delivered content.

It is contemplated that the collection, disclosure, transfer, analysis, storage, or other use of personal information data should comply with well-established privacy policies or practices. Privacy policies and practices that are generally recognized as meeting or exceeding industry or governmental requirements for maintaining personal information data private and secure should be implemented and consistently used. These policies should be easily accessible and updated as the collection or use of the personal information data changes. Personal information data should be collected for legitimate and reasonable uses and not shared or sold outside of those legitimate uses. The collection or sharing should occur after receipt of the user's informed consent. Additional steps to safeguard and secure access to personal information data and to ensure that others with access to the personal information data adhere to their privacy policies and procedures should be considered. An evaluation by third parties to certify adherence to well-established privacy policies and practices may be performed. Policies and practices should be tailored to the particular types of personal information data being collected or accessed and adapted to applicable laws and standards, including jurisdiction-specific considerations. For example, the collection of or access to certain health data in the US may be governed by federal or state laws, such as the Health Insurance Portability and Accountability Act (HIPAA); whereas the collection of or access to the same health data may be subject to other regulations and policies in other countries. As such, different privacy practices should be implemented for different types of personal information data in each country.

It is contemplated that, in some instances, users may selectively prevent the use of, or access to, personal information data. Hardware or software features may be provided to prevent or block access to personal information data. For example, in the case of providing feedback content during a meditation, the present technology may be configured to allow users to “opt in” or “opt out” of the collection of personal information data during registration or anytime thereafter. In another example, users can select not to provide physiological data for targeted content delivery services during a meditation. In yet another example, users can select to limit the length of time of providing feedback content during a meditation session is maintained or entirely prohibit monitoring the user. The present technology may also provide notifications relating to the access or use of personal information data. For example, a first notification may be provided in response to a user downloading an app that may access the user's personal information data and a second notification may be provided to remind the user just before the app accesses the personal information data.

Personal information data should be managed and handled to reduce the risk of unintentional or unauthorized access or use. Risk can be reduced by limiting the collection of data and deleting the data once it is no longer needed. When applicable, data de-identification may be used to protect a user's privacy. For example, de-identification may be performed by removing specific identifiers, controlling the specificity or amount of data stored (e.g., collecting home location data at a city level instead of at an address level), controlling how data is stored (e.g., aggregate data across multiple users), or by using other techniques.

Although the present technology may broadly include the use of personal information data, it may be implemented without accessing such personal information data. In other words, the present technology may not be rendered inoperable due to the lack of some or all of such personal information data. For example, feedback content for a meditation can be selected and delivered to users by inferring preferences based on non-personal information data, a reduced amount of personal information data, or publicly available information.

In general, one innovative aspect of the subject matter described in this specification can be embodied in methods that include the actions of, at an electronic device having a processor, obtaining physiological data associated with a user via one or more sensors, determining that an attentive state is mind wandering during a meditation, the attentive state determined based on the physiological data, customizing feedback content to guide the user to change the attentive state during the meditation, wherein the characteristic is customized based on an user attribute, and providing, after a delay time based on the user attribute, the customized feedback content during the meditation.

These and other embodiments can each optionally include one or more of the following features.

In some aspects, the method further includes, in response to providing the customized feedback content, determining a level of attentiveness corresponding to the user based on the physiological data for a period of time, and determining a feedback metric for the user based on the determined level of attentiveness for the period of time.

In some aspects, the user attribute includes a meditation experience level. In some aspects, the meditation experience level is determined based on accessing a user profile. In some aspects, the meditation experience level is determined based on an analysis of historical data associated with the user for prior meditation experiences. In some aspects, the meditation experience level is updated based on the attentive state during the meditation.

In some aspects, the method further includes presenting an instruction for the user to be attentive to breathing, and assessing a level of attentiveness to breathing based on a respiratory state and the attentive state, where the customized feedback is determined based on the level of attentiveness.

In some aspects, customizing the characteristic of the feedback content includes determining a baseline corresponding to the user based on the physiological data, determining a goal for the user based on the baseline, and determining the customized feedback content based on the baseline and the goal.

In some aspects, the method further includes identifying meditation states of the user corresponding to multiple periods of time, and presenting indications of progress based on the meditation states. In some aspects, the method further includes identifying a portion of the meditation associated with a particular attentive state. In some aspects, the method further includes determining a context of the meditation based on sensor data of an environment of the meditation, and customizing the characteristic of the feedback content based on the context of the meditation.

In some aspects, determining the context of the experience includes generating a scene understanding of the environment based on the sensor data of the environment, the scene understanding including visual or auditory attributes of the environment, and determining the context of the experience based on the scene understanding of the environment. In some aspects, determining the context of the experience includes determining an activity of the user based on a user's schedule.

In some aspects, the customized feedback content includes conclusion of a current meditation, initiation of another meditation different than the current meditation, or changing of the current meditation. In some aspects, the customized feedback content includes a volume of an audio signal that is modulated based on the physiological data. In some aspects, the customized feedback content includes a visual or audible representation of the attentive state or a change to the attentive state. In some aspects, the customized feedback content includes a cue configured to trigger a change in the attentive state. In some aspects, the customized feedback content includes a graphical indicator or sound configured to change a first attentive state to a second attentive state. In some aspects, the customized feedback content includes a visual or audible indication of a suggested time for a new meditation experience.

In some aspects, the attentive state is determined based on using the physiological data to measure gaze or body stability. In some aspects, the attentive state is determined based on determining a level of attentiveness. In some aspects, the attentive state is determined based on a respiratory state. In some aspects, the attentive state is assessed using a statistical or machine learning-based classification technique.

In some aspects, the physiological data includes at least one of skin temperature, respiration, photoplethysmogram (PPG), electrodermal activity (EDA), eye gaze tracking, and pupillary movement that is associated with the user.

In some aspects, an environment of the meditation includes an extended reality (XR) environment. In some aspects, the meditation is presented to multiple users during a communication session. In some aspects, the device is a head-mounted device (HMD).

In accordance with some implementations, a non-transitory computer readable storage medium has stored therein instructions that are computer-executable to perform or cause performance of any of the methods described herein. In accordance with some implementations, a device includes one or more processors, a non-transitory memory, and one or more programs; the one or more programs are stored in the non-transitory memory and configured to be executed by the one or more processors and the one or more programs include instructions for performing or causing performance of any of the methods described herein.

### BRIEF DESCRIPTION OF THE DRAWINGS

So that the present disclosure can be understood by those of ordinary skill in the art, a more detailed description may be had by reference to aspects of some illustrative implementations, some of which are shown in the accompanying drawings.

FIG. 1 illustrates a device presenting a visual and/or auditory experience and obtaining physiological data from a user in accordance with some implementations.

FIG. 2 illustrates a pupil of the user of FIG. 1 in which the diameter of the pupil varies with time in accordance with some implementations.

FIGS. 3A and 3B illustrate detecting a respiratory state and an attention state of the user viewing content based on physiological data in accordance with some implementations.

FIG. 4 illustrates a user viewing content based on tracking the user's respiratory state and attention state in accordance with some implementations.

FIGS. 5A-5C illustrate exemplary views of an electronic device viewing an XR environment, in accordance with some implementations.

FIG. 6 illustrates a system diagram for assessing a respiratory state and an attention state of the user viewing content based on physiological data in accordance with some implementations.

FIG. 7 is a flowchart representation of a method for assessing a respiratory state and an attention state of a user viewing content based on physiological data, and providing content modifications based on the respiratory state and the attention state of the user in accordance with some implementations.

FIG. 8 is a flowchart representation of a method for providing customized feedback content during meditation based on physiological data in accordance with some implementations.

FIG. 9 illustrates device components of an exemplary device in accordance with some implementations.

FIG. 10 illustrates an example head-mounted device (HMD) in accordance with some implementations.

In accordance with common practice the various features illustrated in the drawings may not be drawn to scale. Accordingly, the dimensions of the various features may be arbitrarily expanded or reduced for clarity. In addition, some of the drawings may not depict all of the components of a given system, method or device. Finally, like reference numerals may be used to denote like features throughout the specification and figures.

### DESCRIPTION

Numerous details are described in order to provide a thorough understanding of the example implementations shown in the drawings. However, the drawings merely show some example aspects of the present disclosure and are therefore not to be considered limiting. Those of ordinary skill in the art will appreciate that other effective aspects or variants do not include all of the specific details described herein. Moreover, well-known systems, methods, components, devices and circuits have not been described in exhaustive detail so as not to obscure more pertinent aspects of the example implementations described herein.

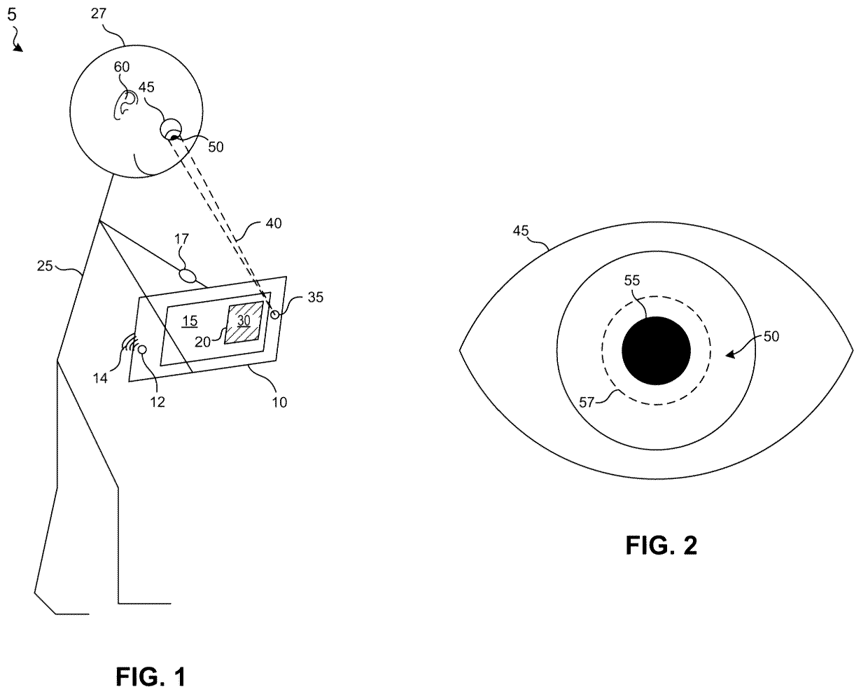

FIG. 1 illustrates a real-world physical environment 5 including a device 10 with a display 15. In some implementations, the device 10 displays content 20 to a user 25, and a visual characteristic 30 that is associated with content 20. For example, content 20 may be a button, a user interface icon, a text box, a graphic, etc. In some implementations, the visual characteristic 30 associated with content 20 includes visual characteristics such as hue, saturation, size, shape, spatial frequency, motion, highlighting, etc. For example, content 20 may be displayed with a visual characteristic 30 of a color highlighting covering or surrounding content 20.

In some implementations, content 20 may be a visual experience (e.g., a meditation experience), and the visual characteristic 30 of the visual experience may continuously change during the visual experience. As used herein, the phrase “experience” refers to a period of time during which a user uses an electronic device that measures one or more respiratory state and/or attention state using physiological data streams. In one example, a user has an experience in which the user perceives a real-world physical environment while holding, wearing, or being proximate to an electronic device that includes one or more sensors that obtain physiological data that is indicative of the user's respiratory state and attention state. In another example, a user has an experience in which the user perceives content displayed by an electronic device while the same or another electronic obtains physiological data (e.g., pupil data, EEG data, etc.) to assess the user's respiratory state and attention state. In another example, a user has an experience in which the user holds, wears, or is proximate to an electronic device that provides a series of audible or visual instructions that guide the experience. For example, the instructions may instruct the user to maintain or try to maintain a particular respiratory state (e.g., 7 breaths per minute (BPM)) and attention state (e.g., focus on a particular visual and/or audio element) during particular time segments of the experience. For example, instructing the user to focus on his or her breathing and paying attention to a particular portion of a meditation video, etc. During such an experience, the same or another electronic device may obtain physiological data from one or more sensors to assess the user's respiratory state and attention state.

In some implementations, the visual characteristic 30 is a content enhancement or content modification for the user that is specific to the experience (e.g., a visual or audio cue to focus on a particular task during an experience, such as paying attention during a particular part of a meditation or education/learning experience). In some implementations, the visual experience (e.g., content 20) can occupy the entire display area of display 15. For example, during a meditation experience, content 20 may be a video or sequence of images that may include visual and/or audio cues as the visual characteristic 30 presented to the user to pay attention to a particular meditation technique. Other visual experiences that can be displayed for content 20 and visual and/or audio cues for the visual characteristic 30 will be further discussed herein.

The device 10 obtains physiological data (e.g., EEG amplitude/frequency, pupil modulation, eye gaze saccades, etc.) from the user 25 via a sensor 35. For example, the device 10 obtains pupillary data 40 (e.g., eye gaze characteristic data). While this example and other examples discussed herein illustrate a single device 10 in a real-world physical environment 5, the techniques disclosed herein are applicable to multiple devices and multiple sensors, as well as to other real-world environments/experiences. For example, the functions of device 10 may be performed by multiple devices.

In some implementations, as illustrated in FIG. 1, the device 10 is a handheld electronic device (e.g., a smartphone or a tablet). In some implementations the device 10 is a laptop computer or a desktop computer. In some implementations, the device 10 has a touchpad and, in some implementations, the device 10 has a touch-sensitive display (also known as a “touch screen” or “touch screen display”). In some implementations, the device 10 is a wearable head mounted display (HMD).

In some implementations, the device 10 includes an eye tracking system for detecting eye position and eye movements. For example, an eye tracking system may include one or more infrared (IR) light-emitting diodes (LEDs), an eye tracking camera (e.g., near-IR (NIR) camera), and an illumination source (e.g., an NIR light source) that emits light (e.g., NIR light) towards the eyes of the user 25. Moreover, the illumination source of the device 10 may emit NIR light to illuminate the eyes of the user 25 and the NIR camera may capture images of the eyes of the user 25. In some implementations, images captured by the eye tracking system may be analyzed to detect position and movements of the eyes of the user 25, or to detect other information about the eyes such as pupil dilation or pupil diameter. Moreover, the point of gaze estimated from the eye tracking images may enable gaze-based interaction with content shown on the near-eye display of the device 10. Additional camera's may be included to capture other areas of the user (e.g., an HMD with a jaw cam to view the user's mouth, a down cam to view the body, an eye cam for tissue around the eye, and the like). These cameras and other sensors can detect motion of the body, and/or signals of the face modulated by the breathing of the user (e.g., remote PPG).

In some implementations, the device 10 has a graphical user interface (GUI), one or more processors, memory and one or more modules, programs or sets of instructions stored in the memory for performing multiple functions. In some implementations, the user 25 interacts with the GUI through finger contacts and gestures on the touch-sensitive surface. In some implementations, the functions include image editing, drawing, presenting, word processing, website creating, disk authoring, spreadsheet making, game playing, telephoning, video conferencing, e-mailing, instant messaging, workout support, digital photographing, digital videoing, web browsing, digital music playing, and/or digital video playing. Executable instructions for performing these functions may be included in a computer readable storage medium or other computer program product configured for execution by one or more processors.

In some implementations, the device 10 employs various sensor, detection, or measurement systems. Detected physiological data may include, but is not limited to, EEG, electrocardiography (ECG), electromyography (EMG), functional near infrared spectroscopy signal (fNIRS), blood pressure, skin conductance, or pupillary response. The device 10 maybe communicatively coupled to an additional sensor. For example, sensor 17 (e.g., an EDA sensor) maybe communicatively coupled to device 10 via a wired or wireless connection, and sensor 17 may be located on the skin of the user 25 (e.g., on the arm as illustrated, or placed on the hand/fingers of the user). For example, sensor 17 can be utilized for detecting EDA (e.g., skin conductance), heart rate, or other physiological data that utilizes contact with the skin of a user. Moreover, the device 10 (using one or more sensors) may concurrently detect multiple forms of physiological data in order to benefit from synchronous acquisition of physiological data. Moreover, in some implementations, the physiological data represents involuntary data, e.g., responses that are not under conscious control. For example, a pupillary response may represent an involuntary movement.

In some implementations, one or both eyes 45 of the user 25, including one or both pupils 50 of the user 25 present physiological data in the form of a pupillary response (e.g., pupillary data 40). The pupillary response of the user 25 results in a varying of the size or diameter of the pupil 50, via the optic and oculomotor cranial nerve. For example, the pupillary response may include a constriction response (miosis), e.g., a narrowing of the pupil, or a dilation response (mydriasis), e.g., a widening of the pupil. In some implementations, the device 10 may detect patterns of physiological data representing a time-varying pupil diameter.

In some implementations, a pupillary response may be in response to auditory feedback that one or both ears 60 of the user 25 detect (e.g., an audio notification to the user). For example, device 10 may include a speaker 12 that projects sound via sound waves 14. The device 10 may include other audio sources such as a headphone jack for headphones, a wireless connection to an external speaker, and the like.

FIG. 2 illustrates a pupil 50 of the user 25 of FIG. 1 in which the diameter of the pupil 50 varies with time. Pupil diameter tracking may be potentially indicative of a physiological state of a user. As shown in FIG. 2, a present physiological state (e.g., present pupil diameter 55) may vary in contrast to a past physiological state (e.g., past pupil diameter 57). For example, the present physiological state may include a present pupil diameter and a past physiological state may include a past pupil diameter.

The physiological data may vary in time and the device 10 may use the physiological data to measure one or both of a user's physiological response to the visual characteristic 30 or the user's intention to interact with content 20. For example, when presented with content 20, such as a list of content experiences (e.g., meditation environments), by a device 10, the user 25 may select an experience without requiring the user 25 to complete a physical button press. In some implementations, the physiological data may include the physiological response of a visual or an auditory stimulus of a radius of the pupil 50 after the user 25 glances at content 20, measured via eye-tracking technology (e.g., via an HMD). In some implementations, the physiological data includes EEG amplitude/frequency data measured via EEG technology, or EMG data measured from EMG sensors or motion sensors.

People may sense or interact with a physical environment or world without using an electronic device. Physical features, such as a physical object or surface, may be included within a physical environment. For instance, a physical environment may correspond to a physical city having physical buildings, roads, and vehicles. People may directly sense or interact with a physical environment through various means, such as smell, sight, taste, hearing, and touch. This can be in contrast to an extended reality (XR) environment that may refer to a partially or wholly simulated environment that people may sense or interact with using an electronic device. The XR environment may include virtual reality (VR) content, mixed reality (MR) content, augmented reality (AR) content, or the like. Using an XR system, a portion of a person's physical motions, or representations thereof, may be tracked and, in response, properties of virtual objects in the XR environment may be changed in a way that complies with at least one law of nature. For example, the XR system may detect a user's head movement and adjust auditory and graphical content presented to the user in a way that simulates how sounds and views would change in a physical environment. In other examples, the XR system may detect movement of an electronic device (e.g., a laptop, tablet, mobile phone, or the like) presenting the XR environment. Accordingly, the XR system may adjust auditory and graphical content presented to the user in a way that simulates how sounds and views would change in a physical environment. In some instances, other inputs, such as a representation of physical motion (e.g., a voice command), may cause the XR system to adjust properties of graphical content.

Numerous types of electronic systems may allow a user to sense or interact with an XR environment. A non-exhaustive list of examples includes lenses having integrated display capability to be placed on a user's eyes (e.g., contact lenses), heads-up displays (HUDs), projection-based systems, head mountable systems, windows or windshields having integrated display technology, headphones/earphones, input systems with or without haptic feedback (e.g., handheld or wearable controllers), smartphones, tablets, desktop/laptop computers, and speaker arrays. Head mountable systems may include an opaque display and one or more speakers. Other head mountable systems may be configured to receive an opaque external display, such as that of a smartphone. Head mountable systems may capture images/video of the physical environment using one or more image sensors or capture audio of the physical environment using one or more microphones. Instead of an opaque display, some head mountable systems may include a transparent or translucent display. Transparent or translucent displays may direct light representative of images to a user's eyes through a medium, such as a hologram medium, optical waveguide, an optical combiner, optical reflector, other similar technologies, or combinations thereof. Various display technologies, such as liquid crystal on silicon, LEDs, uLEDs, OLEDs, laser scanning light source, digital light projection, or combinations thereof, may be used. In some examples, the transparent or translucent display may be selectively controlled to become opaque. Projection-based systems may utilize retinal projection technology that projects images onto a user's retina or may project virtual content into the physical environment, such as onto a physical surface or as a hologram.

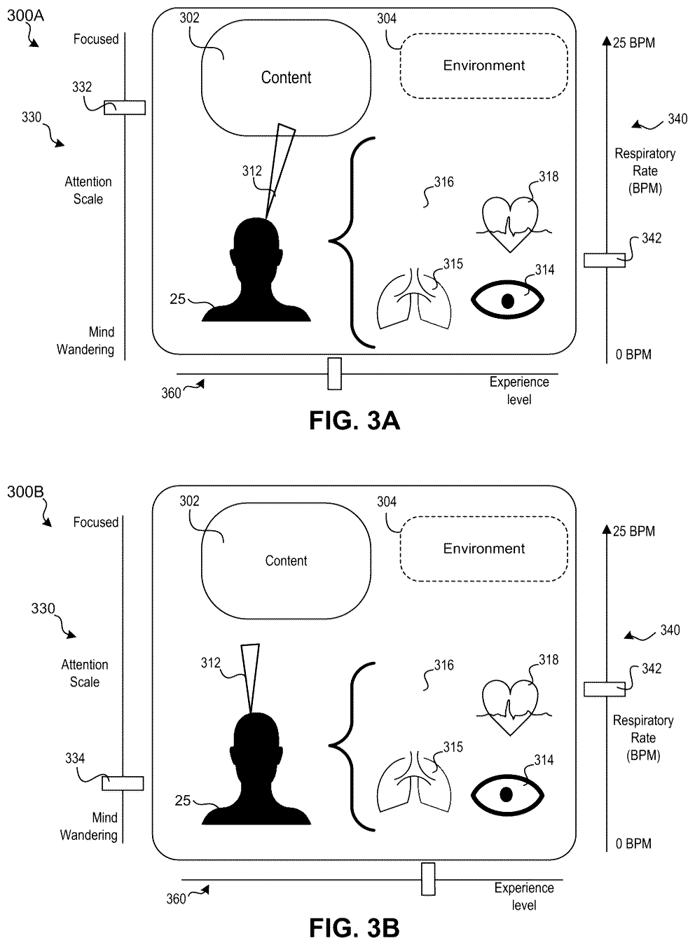

FIG. 3A and FIG. 3B illustrate assessing a respiratory state and an attention state of a user viewing content based on obtained physiological data in accordance with some implementations. In particular, FIGS. 3A and 3B illustrate a user (e.g., user 25 of FIG. 1) being presented with content 302 in an environment 304 during a content presentation (e.g., a meditation experience) at content presentation instant 300A and later in time at content presentation instant 300B, respectively, where the user, via obtained physiological data, has a physiological response to the content (e.g., the user looks towards portions of the content as detected by eye gaze characteristic data). For example, a user is being presented with content 302 that includes visual content (e.g., a meditation video), and the user's physiological data such as eye gaze characteristic data 312, pupillary data 314, respiratory data 315, EDA data 316, and heart rate data 318 are continuously (or periodically) monitored. The physiological data may initially be obtained to determine a user's baseline data, then during an experience (e.g., a mediation session), the physiological data can be monitored and compared to the determined baseline to assess the respiratory state and the attention state of the user.

In the particular examples of FIGS. 3A and 3B, at content presentation instant 300A, the user's eye gaze characteristic is focused on the content 302, such that the attention scale 330 shows the sliding bar indicator 332 as higher towards the “focused” portion, and a lower respiratory rate (e.g., via respiratory scale 340 showing the sliding bar indicator 342 at a lower rate). Then, at content presentation instant 300B of FIG. 3B (e.g., during a mind wandering stage), the user's eye gaze characteristic 312 appears to not be focused on the content 302, such that the attention scale 330 shows the sliding bar indicator 332 as lower towards the “mind wandering” portion, and a higher respiratory rate on the respiratory scale 340 then at content presentation instant 300A.

In some implementations, the respiratory state (e.g., via respiratory scale 340) is based on the acquired respiratory data 315 from a respiratory sensor (e.g., a sensor worn on the user). Additionally, or alternatively, respiratory data 315 may involve sensor fusion of different acquired data from device 10, without using an additional respiratory sensor. For example, the different acquired data that may be fused may include head pose data from an IMU, audio from a microphone, camera images of the user's face and/or body (e.g., an HMD with a jaw cam, down cam to view the body, eye cam for tissue around the eye, and the like), motion of the body, and/or signal of the face modulated by the breath (e.g., remote PPG). Using this type of sensor fusion to track the breathing of the user, such as while wearing an HMD, may negate the need for a user to wear a sensor worn around the user's diaphragm, for example, to track his or her respiratory rates.

In some implementations, the respiratory state and the attention state of the user viewing content is assessed based on physiological data and context data. For example, the content 302 may be analyzed by a context analysis instruction set to determine context data for the experience of the user (e.g., the experience of being present in the current physical environment while watching video content on an electronic device such as an HMD). Determining context data of the experience may involve using computer vision to generate a scene understanding of the visual and/or auditory attributes of the physical environment (e.g., environment 304), such as where is the user, what is the user doing, what objects are nearby. Additionally, or alternatively, determining context data of the experience may involve determining a scene understanding of the visual and/or auditory attributes of the content presentation (e.g., content 302, such as a video). For example, content 302 and environment 304 may include one or more people, objects, or other background objects that are within view of the user that may be detected by an objection detection algorithm, face detection algorithm, or the like.

In some implementations, an experience level (e.g., a meditation experience level) of the user may be determined based on historical data such as physiological data and context data from a prior experience. Additionally, or alternatively, an experience level (e.g., a meditation experience level) of the user may be determined based on accessing a user profile (e.g., storing a metric in a database). For example, the experience level scale 360 indicates the user at content presentation instant 300B has a higher experience level than at content presentation instant 300A at a prior experience. For example, after a successful meditation experience (e.g., content presentation instant 300A) the user's “experience” in successfully meditating has increased. The scale may be a metric based on an amount of time spent in a “focused” attentive state during a meditation experience or another type of experience, or may include additional factors and/or metrics.

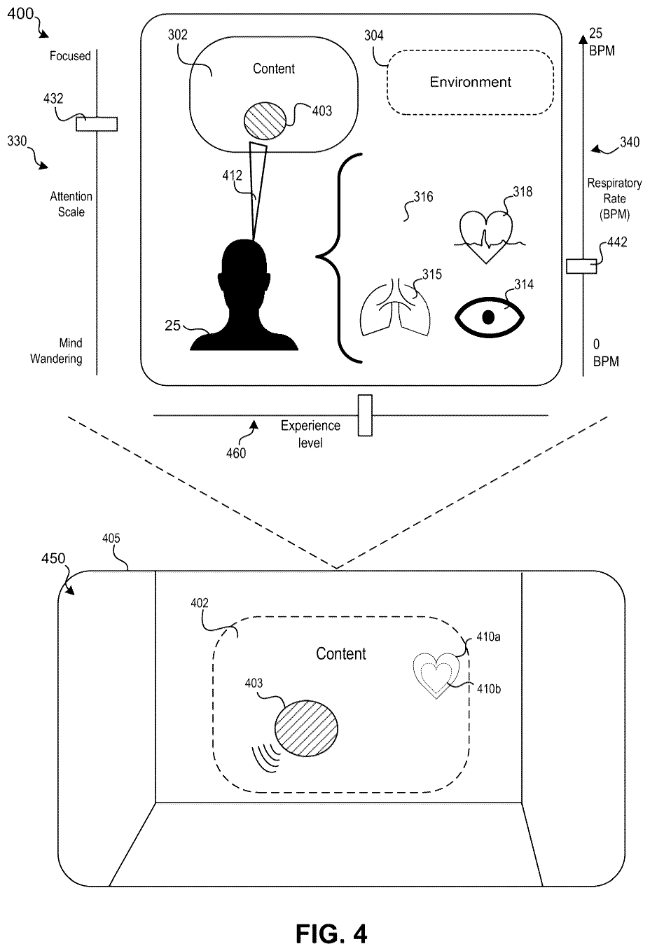

FIG. 4 illustrates assessing a respiratory state and an attention state of a user viewing content and modifying the content based on obtained physiological data, in accordance with some implementations. The user is being presented with content 302 at content presentation instant 400 (e.g., after content presentation instant 300B) that includes visual content (e.g., a meditation video), and the user's physiological data such as eye gaze characteristic data 412, pupillary data 314, respiratory data 315, EDA data 316, and heart rate data 318 are continuously or periodically monitored. In particular, FIG. 4 illustrates a user (e.g., user 25 of FIG. 1) being presented with content 302 in an environment 304 during a content presentation (e.g., a meditation experience) at content presentation instant 400 where the user, via obtained physiological data, has a physiological response to the content. For example, the user looks towards portions of the content, in particular the content enhancement 403, as detected by eye gaze characteristic data 412, where the content is modified based on that response. For example, after content presentation instant 300B, where the user was determined to be “mind wandering”, a content enhancement 403 is applied to the content 302. Thus, after a segment of time after the user's physiological data is analyzed (e.g., by a physiological data instruction set) and the context data of the content 302 and/or environment 304 is analyzed (e.g., by a context instruction set), content presentation instant 400 is presented to the user with a content enhancement 403 because the respiratory state and/or the attention state assessment was that the user may have exhibited higher respiratory state and/or an undesired attention state (e.g., mind wandering). FIG. 4 further illustrates the user is more “attentive” based on the attention scale 330, where the attention sliding bar indicator 432 is higher on the scale than at content presentation instant 300B when the user was mind wandering. Additionally, the user's respiratory rate is shown on the respiratory rate scale 340, where the respiratory sliding bar indicator 442 indicates a breathing rate of about 7 BPM. Moreover, the user's experience level is shown on the experience level scale 460 (e.g., a meditation level).

FIG. 4 further illustrates an exemplary view 450 of a physical environment (e.g., real-world environment 5 of FIG. 1) provided by electronic device 405 (e.g., device 10). The view 450 may be a live camera view of the physical environment, a view of the physical environment through a see-through display, or a view generated based on a 3D model corresponding to the physical environment. The view 450 includes an application window presented on the device of content 402 (e.g., a representation of content 302). The presentation of content 402 includes the content enhancement 403 (e.g., an audio and/or visual notification). Additionally, view 450 includes a visual respiratory indicator 410 that provides the user an indication of the user's respiratory rate (e.g., as detected by the obtained physiological data—respiratory data 315). In particular, respiratory indicator 410a represents a user inhaling (e.g., taking a deep breath in thus a larger icon), and respiratory indicator 410b represents a user exhaling (e.g., taking a deep breath out thus a smaller icon). The visual respiratory indicator 410 can be used in a meditation experience to guide the user to proper breathing techniques. Alternatively, other visual indicators may be used to visualize for the user the respiratory rate (both actual and desired). For example, a sinusoidal wave of the user's respiratory rate may be shown, along with a desired sinusoidal wave that the user can try and mimic (e.g., slowing the user's respiratory rate for a particular meditation experience).

For example, a user is exhibiting a high respiratory state while at work, the content enhancement 403 may indicate the high respiratory state to the user (e.g., an audio and/or visual notification) and may provide the user with some alternative actions for calming down (e.g., meditation music, a relaxing XR environment, etc.). As illustrated, the respiratory scale 340 and the attention state scale 330 provides a possible use case of comparing the detected level of attention and respiratory rate of the user and the performance level associated with those states. For example, for the above example of exhibiting an abnormal respiratory state and attention state (e.g., a focused or distracted level), then the content enhancement 403 could alert the user of the abnormal state to try and instruct him or her to a level within a threshold of the current experience (e.g., control the user to a controlled breath rate, e.g., 7 BPM). Additionally, the user's respiratory state and the attention state assessment can be continuously monitored throughout the presentation of the content 402.

The content enhancement 403 may include a visual presentation. For example, an icon may appear, or a text box may appear instructing the user to pay attention. In some implementations, the content enhancement 403 may include an auditory stimulus. For example, spatialized audio may be presented to redirect the user's attention towards the particular areas of the content presentation (e.g., if determined the user was exhibiting abnormal respiration levels, could steer the user's attention towards something relaxing in content).

In some implementations, the content enhancement 403 may include an entire display of visual content (e.g., a relaxation video over the entire display of the device). The content (and/or content enhancement 403) may include or provide a view of a 3D environment. Alternatively, the content enhancement 403 may include visual content around the frame of the display of the device (e.g., on a mobile device, a virtual frame of the display be created to acquire the user's attention from a particular respiratory state and the attention state). In some implementations, the content enhancement 403 may include a combination of visual content (e.g., a notification window, an icon, or other visual content described herein) and/or an auditory stimulus. For example, a notification window or arrow may direct the user to a particular content area and an audio signal may be presented that directs the user. These visual and/or auditory cues can help direct the user to particular content enhancements that can aid the user in coping with different respiratory state and the attention states to increase his or her performance levels (for a work experience), or simply for comfortable viewing of the content 302 (e.g., providing meditation if determined the user is in a stressful environment or situation).

In some implementations, the content enhancement may be referred to herein as “customized feedback content” for a meditation experience such as a meditation session. In some implementations, the customized feedback content includes a conclusion of a current meditation, an initiation of another meditation different than the current meditation, and/or changing of the current meditation. In some implementations, the customized feedback content includes a volume of an audio signal that is modulated based on the physiological data. In some implementations, the customized feedback content includes a visual or audible representation of the attentive state or a change to the attentive state. In some implementations, the customized feedback content includes the customized feedback content includes a cue configured to trigger a change in the attentive state. In some implementations, the customized feedback content includes a graphical indicator or sound configured to change a first attentive state to a second attentive state. In some implementations, the customized feedback content includes a visual or audible indication of a suggested time for a new meditation experience.

In some implementations, the content 402 for device 10 is for an auditory only experience. For example, for a driving experience (e.g., a truck driver), physiological data and/or context data for the environment may be analyzed by one or more sensors, and the customized feedback content may provide one or more auditory signals for the content enhancement 403 to signify when the user is losing focus while driving and needs to be alerted of a potentially dangerous situation, such as falling asleep while driving. Thus, the content enhancement 403 could include a voice that alerts the driver (e.g., “wake up!”) or a loud alarm beeping notification. In some implementations, the content 402 may be displayed to the user on a windshield of the vehicle or if the user is wearing an HMD such as pass-through glasses, and a virtual notification may be presented to the user as the content enhancement 403. For example, an AR alert may be displayed to a truck driver to alert them to focus on driving.

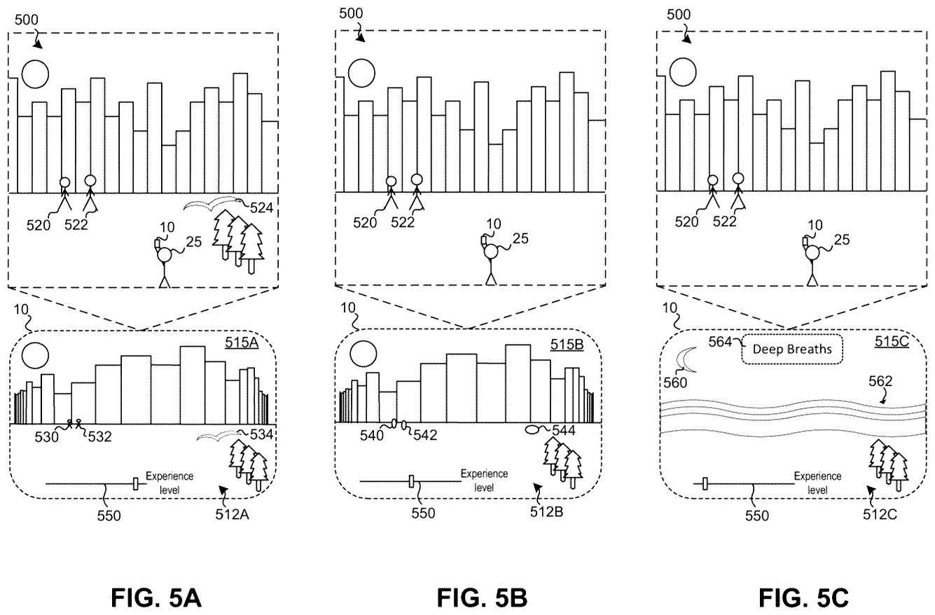

FIGS. 5A-5C illustrate exemplary views of an electronic device viewing an XR environment in accordance with some implementations. In particular, FIGS. 5A-5C illustrate different levels of immersion or displaying different levels of virtual content for a user viewing an XR environment. For instance, FIGS. 5A-5C illustrate an exemplary electronic device 10 providing view 515A of 3D environment 512A, view 515B of 3D environment 512B, and view 515C of 3D environment 512C, respectively, operating in a physical environment 500 during a viewing of content (e.g., a mixed reality meditation experience while walking in a city park). For example, FIGS. 5A-5C may represent a viewing of content at three different periods of time while the user 25 views content on the display of device 10 and may view at least a portion of the physical environment 500. In these examples of FIGS. 5A-5C, the physical environment 500 is a city park with a city (buildings) in the background that includes bystanders 520 and 522 and a bird 524. In particular, FIGS. 5A-5C each illustrate the user 25 (e.g., a viewer during a mediation experience) viewing content on device 10 during a sunrise (e.g., in the morning) in a city park with the city landscape in the background.

The electronic device 10 includes one or more cameras, microphones, depth sensors, or other sensors that can be used to capture information about and evaluate the physical environment 500 and the objects within it, as well as information about the user 25 of the electronic device 10 (e.g., positional data of the user 25). The information about the physical environment 500 and/or user 25 may be used to provide visual and audio content during the meditation session. For example, a meditation session may provide views (e.g., views 515A, 515B, and 515C) of a 3D environment (e.g., 3D environment 512A, 512B, and 512C) that is generated based on camera images and/or depth camera images of the physical environment 100 and, optionally, include virtual content as part of the meditation experience to simulate a nature walk in the city park, but to supplement some (or all) of the physical content with virtual content.

In the example illustrated in FIG. 5A, the electronic device 10 provides a view 515A that includes a representation 530 of bystander 520, representation 532 of bystander 522, representation 534 of bird 524. The view 515A further includes an experience level indicator 550 that may be displayed to the user 25 during the experience and may allow a user to change one or more attributes associated with the view 515A to improve the meditation level such as the level of immersion (e.g., blocking out potential distractions, changing the weather, time of day (sunrise/sunset/moon), and the like). Alternatively, the experience level indicator is not displayed to the user. FIG. 5A provides a view 515A that mimics the environment 500, that is, shows a view that includes a physical environment without supplemented virtual content for the meditation experience (e.g., shows the representations 530, 532, and 534). Similarly, in the example illustrated in FIG. 5B, the electronic device 10 provides a view 515B that includes a representation 540 of bystander 520, representation 542 of bystander 522, representation 544 of bird 524, and the experience level indicator 550. FIG. 5B provides a view 515B that has altered the appearance of the representations 540, 542, 544, of the bystanders 520, 522, and bird 524, respectively. For example, based on the experience level (e.g., meditation experience level, or an immersion level, etc.) the techniques described herein may determine to alter the content feedback displayed to the user while meditating in the city park based on the context of the environment (e.g., bystanders and animals within view that may provide distractions during meditation). In some implementations, the user may select, or the system may automatically, blur out or replace with virtual objects any detected object that may interfere with the user's meditation session. For example, if people are greater than a particular threshold away from the user 25 (e.g., 10 feet or greater) then the system may alter the view of the representations 540, 542, 544 of the bystanders 520, 522, and bird 524, respectively, as shown (e.g., opaque ovals instead of people). Similarly, in the example illustrated in FIG. 5C, the electronic device 10 provides a view 515C that includes a representation 560 of a moon, representation 562 of a calming river, a feedback content element 564, and an interactive indicator 550. FIG. 5C provides a view 515C that has altered the time of day (e.g., now at nighttime), as well as removed some or most of the representations for the view of the physical environment (e.g., representations 540, 542, 544 and the city background). For example, the user 25, while meditating in the city park, may be distracted by several portions of the view of the physical environment 500 while trying to meditate (e.g., based on a meditation experience level). Thus, view 515C displays only portions of the physical environment 500 that may be determined to be a naturalistic landscape (e.g., the trees), and may provide a feedback content element 564 to notify the user to focus on his or her breathing (e.g., “Deep Breaths”). For example, if the user is at a city park with a lake and buildings in the background, then maybe the only portion of the physical environment shown to the user would be the lake, and the buildings may be virtually replaced with a more appropriate background for meditation, such as a mountain in the background.

Additionally, the user may select, or the system may automatically, blur out, replace with virtual objects, or remove any detected object that may interfere with the user's meditation session. For example, in the example of view 515C, if people are greater than a particular threshold away from the user 25 (e.g., 10 feet or greater) then the system may alter the view 515C and remove the representations 540, 542 of the bystanders 520, 522, respectively. Thus, if the bystanders 520 and/or 522 walk closer to the user (e.g., less than 20 feet), or the bird 524 flies close to the user 25, then representations 540, 542, respectively, may start to slowly fade in as they walk closer to user 25 until they reach another threshold (e.g., less than 10 feet), and then the representations 540, 542, may be completely shown, or an actual view of the bystanders 520, 522 may “breakthrough” the view 515 so that user 25 can clearly see that objects are close to them. For example, the representation 534 of the bird 524 may initially be blurred out because it is far away, but if the bird (or any animal) gets to close to the user, the system may automatically adjust the view of the representation 534 of the bird 524 so the user 25 knows an animal may be too close (e.g., a dog running towards the user).

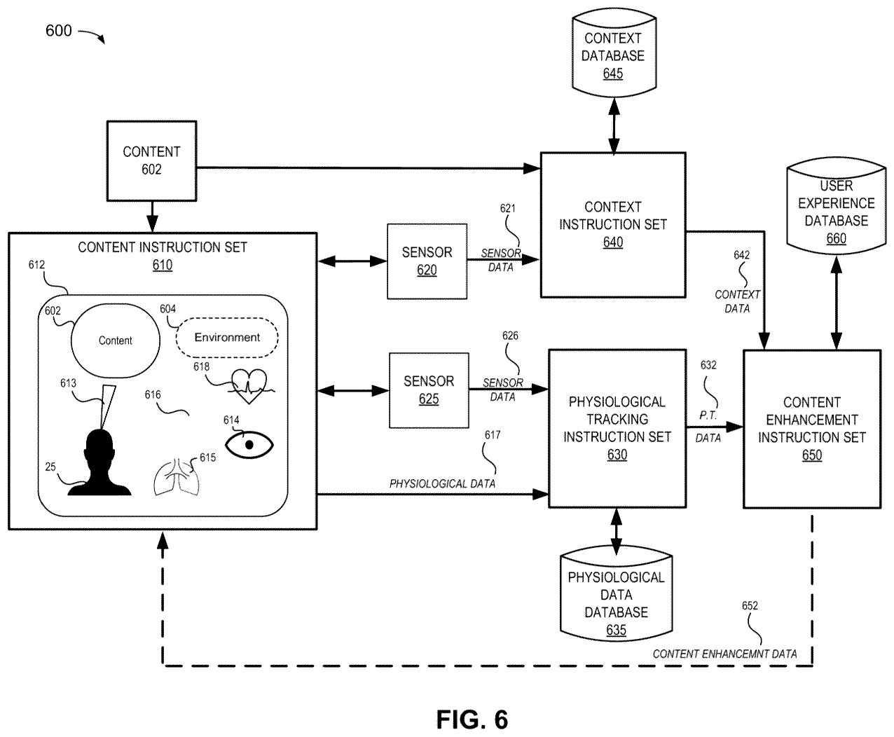

FIG. 6 is a system flow diagram of an example environment 600 in which a respiratory and attentive assessment system can assess a respiratory state and an attention state of a user based on physiological data and provide content enhancement(s) within the presentation of the content according to some implementations. In some implementations, the system flow of the example environment 600 is performed on a device (e.g., device 10 of FIG. 1), such as a mobile device, desktop, laptop, or server device. The content of the example environment 600 can be displayed on a device (e.g., device 10 of FIG. 1) that has a screen (e.g., display 15) for displaying images and/or a screen for viewing stereoscopic images such as an HMD. In some implementations, the system flow of the example environment 600 is performed on processing logic, including hardware, firmware, software, or a combination thereof. In some implementations, the system flow of the example environment 600 is performed on a processor executing code stored in a non-transitory computer-readable medium (e.g., a memory).

The system flow of the example environment 600 acquires and presents content (e.g., video content or a series of image data) to a user (e.g., a meditation experience), obtains physiological data associated with the user during presentation of the content, assesses a respiratory state and the attention state of the user based on the physiological data of the user, and provides a content enhancement based on the respiratory state and the attention state (e.g., a notification/alert based on a distracted threshold). In some implementations, the example environment 600 also analyzes the content and/or the environment for context data and provides a content enhancement based on the respiratory state and the attention state and the context data. For example, a respiratory state and the attention state assessment technique described herein determines, based on obtained physiological data, a user's respiratory state and the attention state during an experience (e.g., watching a meditation video) by providing a content enhancement that is based on the respiratory state and the attention state of the user (e.g., a notification, auditory signal, an alert, an icon, and the like, that alerts the user that they may be at a particular respiratory state and the attention state during the presentation of content).

The example environment 600 includes a content instruction set 610 that is configured with instructions executable by a processor to provide and/or track content 602 for presentation on a device (e.g., device 10 of FIG. 1). For example, the content instruction set 610 provides content presentation instant 612 that includes content 602 to a user 25 while user is within a physical environment 604 (e.g., a room, outside, etc.). For example, content 602 may include background image(s) and sound data (e.g., a video). The content presentation instant 612 could be an XR experience (e.g., a meditation experience) that includes some virtual content and some images of a physical environment (e.g., a meditation experience while viewing a nature scene). Alternatively, the user could be wearing an HMD and is looking at a real physical environment either via a live camera view, or the HMD allows a user to look through the display, such as wearing smart glasses that user can see through, but still be presented visual and/or audio cues. During an experience, while a user 25 is viewing the content 602, tracking of the user's respiratory rate (e.g., respiratory data 615) and pupillary data 614 (e.g., pupillary data 40 such as eye gaze characteristic data) and sent as physiological data 617. Additionally, other physiological data can be monitored and sent as physiological data 617, such as EDA data 616 and heart rate data 618.

The environment 600 further includes a physiological tracking instruction set 630 to track a user's physiological attributes as physiological tracking data 632 using one or more of the techniques discussed herein or as otherwise may be appropriate. For example, the physiological tracking instruction set 630 may acquire physiological data 617 (e.g., pupillary data 614 and respiratory data 615) from the user 25 viewing the content 602. Additionally, or alternatively, a user 25 may be wearing a sensor 625 (e.g., sensor 17 of FIG. 1, such as an EEG sensor, an EDA sensor, heart rate sensor, etc.) that generates sensor data 626 (e.g. EEG data, respiratory data 615, EDA data 616, heart rate data 618) as additional physiological data. Thus, as the content 602 is presented to the user as content presentation instant 612, the physiological data 617 (e.g., pupillary data 614 and respiratory data 615) and/or sensor data 626 is sent to the physiological tracking instruction set 630 to track a user's physiological attributes as physiological tracking data 632, using one or more of the techniques discussed herein or as otherwise may be appropriate. Alternatively, the physiological tracking instruction set 630 obtains physiological data associated with the user 25 from a physiological database 635 (e.g., if the physiological data 617 was previously analyzed by the physiological tracking instruction set, such as during a previously viewed/analyzed video).

In an example implementation, the environment 600 further includes a context instruction set 640 that is configured with instructions executable by a processor to obtain the experience data presented to the user (e.g., content 602) and other sensor data (e.g., image data of the environment 604, the user's 25 face and/or eye's, etc.), and generate context data 642 (e.g., identifying people, objects, etc. of the content 602 and the environment 604). For example, the context instruction set 640 acquires content 602 and sensor data 621 (e.g., image data) from the sensor 620 (e.g., an RGB camera, a depth camera, etc.) and determines context data 642 based on identifying areas of the content while the user is viewing the presentation of the content 602 (e.g., a first time viewed content/video). Sensor 620 and 625 are illustrated as separate blocks (sensors), however, in some implementations, sensor 620 and sensor 625 are the same sensor.

Alternatively, the context instruction set 640 selects context data associated with content 602 from a context database 645 (e.g., if the content 602 was previously analyzed by the context instruction set, such as during a previously viewed/analyzed video). In some implementations, the context instruction set 640 generates a scene understanding associated with content 602 and/or environment 604 as the context data 642. For example, the scene understanding can be utilized to track the overall context of what the user may be focused on during the presentation of content 602, or where the user is, what the user is doing, what physical objects or people are in the vicinity of the user with respect to the environment 604.

In an example implementation, the environment 600 further includes a content enhancement instruction set 650 that is configured with instructions executable by a processor to assess the respiratory state and the attention state of a user based on a physiological response (e.g., eye gaze response, respiratory rates, etc.) using one or more of the techniques discussed herein or as otherwise may be appropriate. For example, the respiratory state and the attention state be assessed by determining where the user's respiratory state and the attention state may be with respect to an indicator, such as the respiratory scale 340 and the attention scale 330 of FIGS. 3 and 4. In particular, the content enhancement instruction set 650 acquires physiological tracking data 632 from the physiological tracking instruction set 630 and context data 642 from the context instruction set 640 (e.g., scene understanding data) and determines the respiratory state and the attention state of the user 25 during the presentation of the content 602 and based on attributes of the physical environment 604 that the user is watching the content 602. For example, the context data 642 may provide a scene analysis that can be used by the content enhancement instruction set 650 to understand what the person is looking at, where they are at, etc., and improve the determination of the respiratory state and the attention state. In some implementations, the content enhancement instruction set 650 can then provide content enhancement data 652 (e.g., visual and/or audible cues) to the content instruction set 610 based on the respiratory state and the attention state assessment. For example, finding defined markers of high/low levels of attention and respiration (e.g., visual respiratory indicator 410), and providing performance feedback during a meditation experience that could enhance a user's meditation experience, provide additional benefits from the meditation session, and provide a guided and supportive teaching approach (e.g., a scaffolding teaching method) for users to advance through their meditation practice.

In some implementations, the content enhancement data 652 could be utilized by the content instruction set 610 to present an audio and/or visual feedback cue or mechanism to the user 25 to relax and focus on breathing during the high-level stress situation (e.g., over anxious about an upcoming test). In an educational experience, the feedback cue to the user could be a gentle reminder (e.g., a soothing or calming visual and/or audio alarm) to get back on task of studying, based on the assessment from the content enhancement instruction set 650 that the user 25 is mind wandering because the user 25 was bored (e.g., a low level attention indication).

In some implementations, the user experience database 660 may be utilized to store information associated with the user 25. For example, historical data may be stored. For example, after each meditation experience, the physiological data 632, context data 642, a meditation experience level, etc., may be monitored and stored in the user experience database 660, before, during, and/or after the customized feedback content (e.g., content enhancement data 652) is presented to the user. In some implementations, the meditation experience level may be determined based on an analysis of historical data associated with the user 25 for prior meditation experiences (e.g., stored in the user experience database 660). Alternatively, in some implementations, the meditation experience level may be determined based on accessing a user profile stored in the user experience database 660.

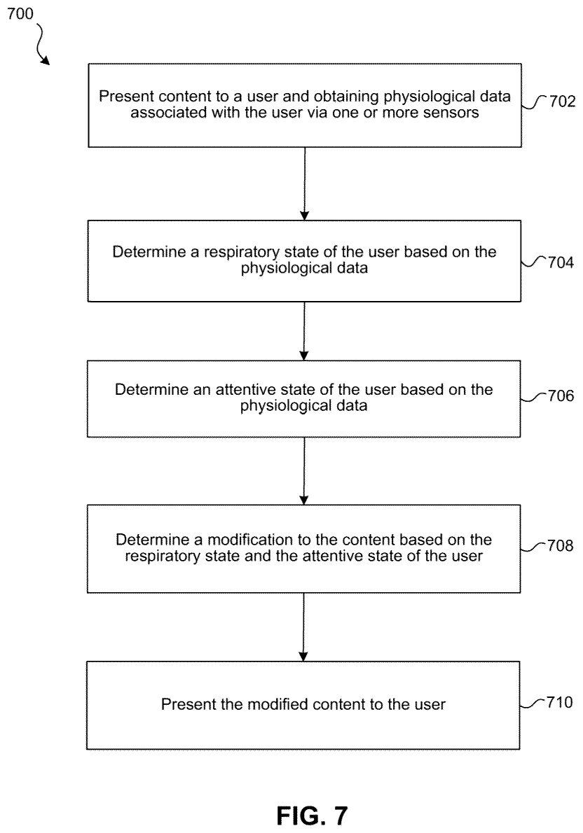

FIG. 7 is a flowchart illustrating an exemplary method 700. In some implementations, a device such as device 10 (FIG. 1) performs the techniques of method 700 to assess a respiratory state and an attention state of the user viewing content based on physiological data, and providing a modification to the content based on the detected respiratory state and the attention state. In some implementations, the techniques of method 700 are performed on a mobile device, desktop, laptop, HMD, or server device. In some implementations, the method 700 is performed on processing logic, including hardware, firmware, software, or a combination thereof. In some implementations, the method 700 is performed on a processor executing code stored in a non-transitory computer-readable medium (e.g., a memory).

At block 702, the method 700 presents content to a user and obtains physiological data associated with the user via one or more sensors. The physiological data may include EEG amplitude/frequency, image data of the user's face, pupil modulation, eye gaze saccades, EDA, heart rate, and the like. For example, obtaining the physiological data may involve obtaining images of the eye or EOG data from which gaze direction/movement can be determined, electrodermal activity/skin conductance, heart rate, via sensors on a watch. Additionally, facial recognition via HMD may be included as physiological data (e.g., reconstruction of the user's face).

In some implementations, obtaining the physiological data associated with a physiological response of the user includes monitoring for a response or lack of response occurring within a predetermined time following the presenting of the content or a user performing a task. For example, the system may wait for up to five seconds after an event within the video to see if a user looks in a particular direction (e.g., a physiological response).

In some implementations, obtaining physiological data (e.g., pupillary data 40) is associated with a gaze of a user that may involve obtaining images of the eye or electrooculography signal (EOG) data from which gaze direction and/or movement can be determined. In some implementations, the physiological data includes at least one of skin temperature, respiration, photoplethysmogram (PPG), electrodermal activity (EDA), eye gaze tracking, and pupillary movement that is associated with the user.

Some implementations obtain physiological data and other user information to help improve a user experience. In such processes, user preferences and privacy should be respected, as examples, by ensuring the user understands and consents to the use of user data, understands what types of user data are used, has control over the collection and use of user data and limiting distribution of user data, for example, by ensuring that user data is processed locally on the user's device. Users should have the option to opt in or out with respect to whether their user data is obtained or used or to otherwise turn on and off any features that obtain or use user information. Moreover, each user will have the ability to access and otherwise find out anything that the system has collected or determined about him or her. User data is stored securely on the user's device. User data that is used as input to a machine learning model is stored securely on the user's device, for example, to ensure the user's privacy. The user's device may have a secure storage area, e.g., a secure enclave, for securing certain user information, e.g., data from image and other sensors that is used for face identification, face identification, or biometric identification. The user data associated with the user's body and/or attention state may be stored in such a secure enclave, restricting access to the user data and restricting transmission of the user data to other devices to ensure that user data is kept securely on the user's device. User data may be prohibited from leaving the user's device and may be used only in machine learning models and other processes on the user's device.

In some implementations, the presented content includes an XR experience (e.g., a meditation session).

At block 704, the method 700 determines a respiratory state of the user based on the physiological data. For example, a machine learning model may be used to determine the respiratory state based on physiological data, and audio/visual content of the experience and/or the environment. For example, one or more physiological characteristics may be determined, aggregated, and used to classify the user's respiratory state using statistical or machine learning techniques. In some implementations, the response may be compared with the user's own prior respiratory responses or typical user respiratory responses to similar content of a similar experience and/or similar environment attributes.

The determined respiratory state may be approximately 7 breaths per minute, as illustrated by the respiratory scale 340 of FIG. 3. In some implementations, determining a respiratory state may involve sensor fusion of different acquired data without using an additional respiratory sensor. For example, the different acquired data that may be fused may include head pose data from an IMU, audio from a microphone, camera images of the user's face and/or body (e.g., an HMD with a jaw cam, down cam, eye cam for tissue around the eye, and the like), motion of the body, and/or signal of the face modulated by the breath (e.g., remote PPG). Using this type of sensor fusion to track the breathing of the user, such as while wearing an HMD, may negate the need for a user to wear a sensor worn around the user's diaphragm, for example, to track his or her respiratory rates.

At block 706, the method 700 determines an attention state of the user based on the obtained physiological data and the context of the experience. For example, a machine learning model may be used to determine the attention state based on eye tracking and other physiological data, and audio/visual content of the experience and/or the environment. For example, one or more physiological characteristics may be determined, aggregated, and used to classify the user's attention state using statistical or machine learning techniques. In some implementations, the response may be compared with the user's own prior responses or typical user responses to similar content of a similar experience and/or similar environment attributes. In some implementations, the attention state is determined based on using the physiological data to measure gaze or body stability. In some implementations, the attention state is determined based on determining a level of attentiveness. In some implementations, the attention state is determined based on the respiratory state (e.g., a particular range of a respiratory rate may indicate the user is focused on a task).

In some implementations, determining that the user has a particular threshold of attention (e.g., high, low, etc.) includes determining a level of attention as a sliding scale. For example, the system could determine a level of attention as an attention barometer that can be customized based on the type of content shown during the user experience. If a high level of attention, if for education, a content developer can design an environment for the experience that will provide the user the “best” environment for a learning experience. For example, tune the ambience lighting so the user can be at the optimal levels to learn during the experience.

In some implementations, respiratory state and attention state may be determined using statistical or machine learning-based classification techniques. For example, determining that the user has a respiratory state and an attention state includes using a machine learning model trained using ground truth data that includes self-assessments in which users labelled portions of experiences with respiratory state and the attention state labels. For example, to determine the ground truth data that includes self-assessments, a group of subjects, while watching meditation video, could be prompted at different time intervals (e.g., every 30 seconds). Alternatively, or additionally, the ground truth data that includes self-assessments while watching a video includes different examples of meditation events. For example, after each “meditation event”, each subject could be prompted at or after a particular meditation event in the video content to enter his or her respiratory state and the attention state.

In some implementations, the method 700 further includes identifying emotional states of the user corresponding to multiple periods of time, and presenting indications of progress based on the emotional states. For example, identifying emotional states of the user maybe based on user input or feedback during the presentation of content (e.g., an emotional log during the meditation experience) and/or identifying emotional states of the user maybe based on the obtained physiological data.

In some implementations, the content is presented to multiple users during a communication session. For example, a couple or a group of people (e.g., 2 or more) may share an experience together in an XR environment (e.g., a video game that monitors a person's physiological data). The shared experience may include a teacher and one or more students, where the teacher (or any other person) can educate the user(s) on ways to improve his or her experience during presentation of the content (e.g., focus on a particular visual and/or audio content, such as a particular content in the game).

In some implementations, one or more pupillary or EEG characteristics may be determined, aggregated, and used to classify the user's respiratory state and attention state using statistical or machine learning techniques. In some implementations, the physiological data is classified based on comparing the variability of the physiological data to a threshold. For example, if the baseline for a user's EEG data is determined during an initial segment of time (e.g., 30-60 seconds), and during a subsequent segment of time following an auditory stimulus (e.g., 5 seconds) the EEG data deviates more than +/−10% from the EEG baseline during the subsequent segment of time, than the techniques described herein could classify the user as transitioned away from the high respiratory state and the attention state and entered a second lower respiratory state and the attention state. Similarly, the heart rate data and/or EDA data may also be classified based on comparing the variability of the heart rate data and/or EDA data to a particular threshold.

In some implementations, the machine learning model is a neural network (e.g., an artificial neural network), decision tree, support vector machine, Bayesian network, or the like. These labels may be collected from the user beforehand, or from a population of people beforehand, and fine-tuned later on individual users. Creating this labeled data may require many users going through an experience (e.g., a meditation experience) where the users listen to natural sounds with intermixed natural-probes (e.g., an auditory stimulus) and then randomly are asked how focused or relaxed they were (e.g., respiratory state and the attention state) shortly after a probe was presented. The answers to these questions can generate a label for the time prior to the question and a deep neural network or deep long short term memory (LSTM) network might learn a combination of features specific to that user or task given those labels (e.g., low respiratory state and attention state, high respiratory state and attention state, etc.).

At block 708, the method 700 determines a modification to the content based on the respiratory state and the attention state of the user, and at block 710, the method 700 presents the modified content to the user. For example, the determined respiratory state and attention state could be used to provide feedback to the user via the content enhancement which may aid the user, provide statistics to the user, and/or help content creators improve the content of the experience. In some aspects, the content enhancement may be the start or end of a meditation experience or an change during an ongoing experience. In some implementations, the modification of the content includes a graphical indicator or sound configured to change a first attention state to a second attention state. In some implementations, the modification of the content includes a visual or audible indication of a suggested time for an experience. In some aspects, the content enhancement may provide a visualization of current respiratory state or desired/improved respiratory state such as oscillations using a shrinking and enlarging icon, such as a heart or a flower (e.g., visual respiratory indicator 410). Alternatively, an interactive icon may be used to encourage a user to breath harder/deeper, such as a virtual candle being blown out. In some aspects, the content enhancement may use subtle cues change from lighter to darker ambience and/or bells, audio, chimes.

In some implementations, the method 700 further includes presenting an instruction for the user to be attentive to breathing and assessing a level of attentiveness to breathing based on the respiratory state and attention state, where the modification is determined based on the level of attentiveness. For example, the content enhancement may escalate explicitness from subtle cues to direct instructions (e.g., “focus on breathing”). In some aspects, the content enhancement may use spatialized audio to redirect attention. In some implementations, the modification includes a visual or audible representation of the respiratory state, and a visual or audible representation of the attention state. In some implementations, the modification includes a cue configured to trigger a change in the respiratory state or attention state.

In some implementations, the method 700 further includes determining a baseline corresponding to the user based on the physiological data, determining a goal for the user based on the baseline, and determining the modification based on the baseline and the goal. For example, in some aspects, the content enhancement may provide simultaneous/combined feedback regarding the respiratory state and the attention state. For example, a sinusoidal wave of the user's respiratory rate may be shown, along with a desired sinusoidal wave that the user can try and mimic (e.g., slowing the user's respiratory rate for a particular meditation experience). In some implementations, during a meditation experience, the modification includes initiation of a new meditation, conclusion of a meditation, or changing of an ongoing meditation.

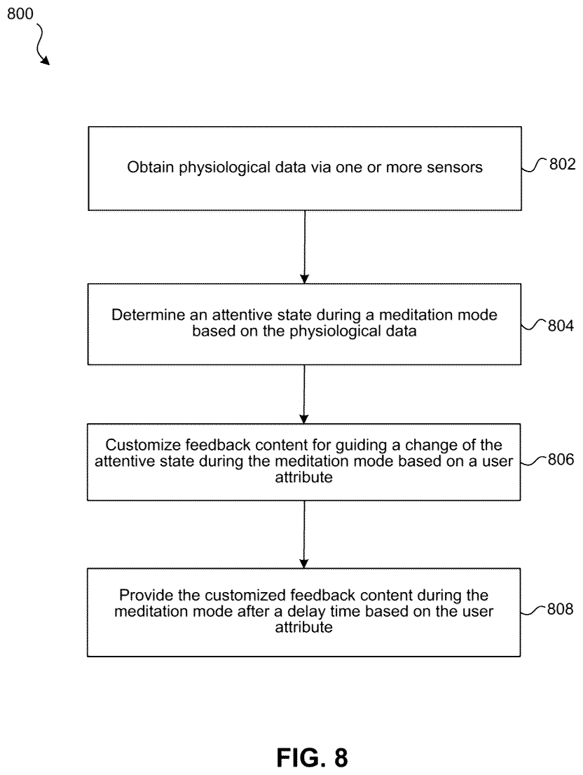

FIG. 8 is a flowchart illustrating an exemplary method 800. In some implementations, a device such as device 10 (FIG. 1) performs the techniques of method 800 to provide customized feedback content during meditation based on physiological data. In some implementations, the techniques of method 800 are performed on a mobile device, desktop, laptop, HMD, or server device. In some implementations, the method 800 is performed on processing logic, including hardware, firmware, software, or a combination thereof. In some implementations, the method 800 is performed on a processor executing code stored in a non-transitory computer-readable medium (e.g., a memory).

At block 802, the method 800 obtains physiological data via one or more sensors. The physiological data may include EEG amplitude/frequency, image data of the user's face, pupil modulation, eye gaze saccades, EDA, heart rate, and the like. For example, obtaining the physiological data may involve obtaining images of the eye or EOG data from which gaze direction/movement can be determined, electrodermal activity/skin conductance, heart rate, via sensors on a watch. Additionally, facial recognition via HMD may be included as physiological data (e.g., reconstruction of the user's face).

At block 804, the method 800 determines an attentive state during a meditation mode based on the physiological data. For example, measuring gaze (e.g., eye gaze stability) may be used to determine that a user is mind wandering because, in a context of breath tracking, for example, mind wandering tends to result in greater eye movement. In some implementations, levels of attention may be monitored as well as overall body stability.

In some implementations, a machine learning model may be used to determine the attention state based on eye tracking and other physiological data, and audio/visual content of the experience and/or the environment. For example, one or more physiological characteristics may be determined, aggregated, and used to classify the user's attention state using statistical or machine learning techniques. In some implementations, the response may be compared with the user's own prior responses or typical user responses to similar content of a similar experience and/or similar environment attributes. In some implementations, the attention state is determined based on using the physiological data to measure gaze or body stability. In some implementations, the attention state is determined based on determining a level of attentiveness. In some implementations, the attention state is determined based on the respiratory state (e.g., a particular range of a respiratory rate may indicate the user is focused on a task).

At block 806, the method 800 customizes feedback content for guiding a change of the attentive state during the meditation mode based on a user attribute. For example, the user attribute may be a meditation experience level, a level of mind wandering, a user's schedule, where the user is, what the user is doing, what objects are near the user based on visual/auditory scene understanding, etc. Additionally, feedback may be customized with respect to how long to delay, what type of feedback to provide, the like.

In some implementations, a heuristic technique is utilized to determine the appropriate amount of delay before trying to help the user refocus (e.g., give them enough time to do it themselves before intervening). In some implementations, the amount of delay before providing customized feedback content may vary based on ability of user (e.g., vary based on the meditation experience level).

In some implementations, the determined attentive state could be used to provide feedback to the user via the customized feedback content which may aid the user, provide statistics to the user, and/or help content creators improve the content of the experience. In some aspects, the customized feedback content may be the start or end of the meditation experience. In some implementations, the modification of the content includes a graphical indicator or sound configured to change a first attention state to a second attention state. In some implementations, the modification of the content includes a visual or audible indication of a suggested time for an experience. In some aspects, the content enhancement may provide a visualization of current respiratory state or desired/improved respiratory state such as oscillations using a shrinking and enlarging icon, such as a heart or a flower (e.g., visual respiratory indicator 410). Alternatively, an interactive icon may be used to encourage a user to breath harder/deeper, such as a virtual candle being blown out. In some aspects, the content enhancement may use subtle cues change from lighter to darker ambience and/or bells, audio, chimes.

At block 808, the method 800 provides the customized feedback content during the meditation mode after a delay time based on the user attribute. In some implementations, the method 800 customizes feedback that encourages a user to refocus to address mind wandering detected during meditation, e.g., when to provide the feedback, how long to delay after mind-wandering is detected before providing feedback, the type of feedback provided, etc. The feedback may be customized based on a user attribute such as the ability or prior experience of the user (e.g., a new user, or an experienced meditator that historically has shown can refocus based on the level of mind wandering). For example, as illustrated in FIGS. 5A-5C, based on the user's experience level of meditation and/or other potential surrounding objects that may detract from a meditation experience (e.g., people, animals, etc.), the techniques described herein may customize the feedback content within a view of the device 10 to improve the user's meditation experience.

In some implementations, the user's experience may be tracked after presenting the customized feedback content during a meditation session. In an exemplary embodiment, the method 800 further includes, in response to providing the customized feedback content, determining a level of attentiveness corresponding to the user based on the physiological data for a period of time, and determining a feedback metric for the user based on the determined level of attentiveness for the period of time. For example, after each meditation experience, the physiological data, context information, meditation experience level, etc. may be monitored and stored (e.g., user experience database 660 in FIG. 6), before, during, and after the customized feedback content is presented to the user. In some implementations, the attribute is a meditation experience level. In some implementations, the meditation experience level may be determined based on an analysis of historical data associated with the user for prior meditation experiences (e.g., stored in the user experience database 660). Alternatively, in some implementations, the meditation experience level may be determined based on accessing a user profile (e.g., stored in the user experience database 660). In some implementations, the meditation experience level may be updated based on the attentive state during the meditation experience.

In some implementations, the feedback content may be customized based on tracking breathing during a meditation session. In an exemplary embodiment, the method 800 further includes presenting an instruction for the user to be attentive to breathing and assessing a level of attentiveness to breathing based on a respiratory state and the attentive state, where the customized feedback is determined based on the level of attentiveness.

In some implementations, customizing the characteristic of the feedback content includes determining a baseline corresponding to the user based on the physiological data, determining a goal for the user based on the baseline, and determining the customized feedback content based on the baseline and the goal. For example, if the baseline for a user's respiratory state is determined during an initial segment of time (e.g., 30-60 seconds of when a user's is focused on a meditation session), and during a subsequent segment of time following some feedback content such as an auditory stimulus (e.g., 5 seconds), if the respiratory data deviates more than +/−10% from the respiratory baseline during the subsequent segment of time, than the techniques described herein could classify the user as transitioned away from the baseline respiratory state (e.g., a lower quality meditation experience).

In some implementations, a meditation experience log may be utilized. In an exemplary embodiment, the method 800 further includes identifying meditation states of the user corresponding to multiple periods of time and presents indications of progress based on the meditation states. For example, based on user input and/or physiological data, meditation states such as emotional states, respiratory levels, etc., may be identified during an experience.

In some implementations, the method 800 further includes identifying a portion of the meditation associated with a particular attentive state. For example, techniques described herein may recommend or not recommend similar content or portions of the content to the user or to help the content developer improve the content for future users (e.g., providing helpful/therapeutic content that has worked for similar experiences from other users for meditation).

In some implementations, a meditation session may be customized based on a context of the environment. In an exemplary embodiment, the method 800 further includes determining a context of the meditation based on sensor data of an environment of the meditation and customizing the characteristic of the feedback content based on the context of the meditation. For example, the customization may be based on using computer vision to generate a scene understanding of the visual and/or auditory attributes of the environment (e.g., where is the user, what is the user doing, what objects are nearby). Additionally, or alternatively, the customization may be based on the video content being presented to user, or what he or she was doing in the environment. In some implementations, determining the context of the experience includes generating a scene understanding of the environment based on the sensor data of the environment, the scene understanding including visual or auditory attributes of the environment, and determining the context of the experience based on the scene understanding of the environment. In some implementations, determining the context of the experience includes determining an activity of the user based on a user's schedule. For example, by accessing a user's calendar, the system can determine that there is a scheduled therapy session.

In some implementations, the customized feedback content includes a conclusion of a current meditation, an initiation of another meditation different than the current meditation, and/or changing of the current meditation. In some implementations, the customized feedback content includes a volume of an audio signal that is modulated based on the physiological data. In some implementations, the customized feedback content includes a visual or audible representation of the attentive state or a change to the attentive state. In some implementations, the customized feedback content includes the customized feedback content includes a cue configured to trigger a change in the attentive state. In some implementations, the customized feedback content includes a graphical indicator or sound configured to change a first attentive state to a second attentive state. In some implementations, the customized feedback content includes a visual or audible indication of a suggested time for a new meditation experience.

In some implementations, the meditation is presented to multiple users during a communication session. For example, a couple or a group of people (e.g., 2 or more) may share a meditation experience together in an XR environment. The shared experience may include an instructor and a patient, where the instructor (or any other person) can educate the user on ways to better meditate during presentation of the content (e.g., focus on particular visual and/or audio content, such as a bird chirping or a waterfall).

The following embodiments or implementations illustrate aspects that method 700, method 800, or any other processes described herein may utilize.

In some implementations, predicting a type of physiological status, such as meditative state (e.g., physical, cognitive, social) may be utilized by the processes described herein. In an exemplary implementation, determining a respiratory state and an attention state of the user during a portion of the experience further includes determining a type of meditation of the user based on the sensor data, and providing the content enhancement during the experience is further based on the type of meditation. For example, if the user is experiencing a low level meditation experience (e.g., mind wandering and not focused on breathing), then the content enhancement may include video and/or auditory content (e.g., a notification to “take a deep breath”, or adding relaxing music) that can aid the user in obtaining a target respiratory state and/or attention state to find a better level of meditative experience.

In some implementations, feedback can be provided to a user based on determining that the respiratory state and the attention state (e.g., playing an intense video game) differs from an intended respiratory state and the attention state of the experience (e.g., content developer wants to increase the respiratory state and the attention state for a particular portion of the video game). In some implementations, the method 700 or 800 may further include presenting feedback (e.g., audio feedback such as “control your breathing”, visual feedback, etc.) during the experience in response to determining that the respiratory state and the attention state differs from a second respiratory state and the attention state intended for the experience. In one example, during a portion of an educational experience in which a user is studying for a difficult test, the method determines to present feedback directing the user to focus on breathing based on detecting that the user is instead in a high respiratory state and the attention state while studying.

In some implementations, the respiratory state and the attention state is a first respiratory state and a first attention state, and the method further includes obtaining, using a sensor, first physiological data (e.g., EEG amplitude, pupil movement, etc.) associated with a physiological response (or lack of response) of the user to the content enhancement, and determining a second respiratory state and second attention state of the user based on the physiological response of the user to the content enhancement. In some implementations, the method further includes assessing the second respiratory state and the second attention state of the user based on the physiological response of the user to the content enhancement, and determining whether the content enhancement reduced stress of the user by comparing the second respiratory state and the second attention state to the first respiratory state and the first attention state. For example, the respiratory state and the attention states may be compared with the user's own prior responses or typical user responses to similar stimuli. The respiratory state and the attention state may be determined using statistical or machine learning-based classification techniques. Additionally, the determined respiratory state and the attention states could be used to provide feedback to the user/reorient the user, provide statistics to the user, or help content creators in creating a more effective meditation experience, learning experience, breathing, workday, and the like.

In some implementations, providing the content enhancement includes providing a graphical indicator or sound configured to change the respiratory state and the attention state to a second respiratory state and the second attention state corresponding to physiological data exhibited by the user in the task during the portion of the experience. In some implementations, providing the content enhancement includes providing a mechanism for rewinding or providing a break from content associated with the task (e.g., rewinding during a cooking video to replay the last step(s), or pausing an educational lecture for a study break). In some implementations, providing the content enhancement includes suggesting a time for another experience based on respiratory state and attention state.

In some implementations, a context analysis may be obtained or generated, to determine what content the user is focusing on that is creating an increase (or decrease) in respiratory state and attention state, which may include a scene understanding of the content and/or the physical environment. In an exemplary implementation, the method 700 or 800 may further include identifying the portion of the experience associated with the respiratory state and the attention state. For example, identifying a portion of the experience associated with a particularly high respiratory state and/or low attention state (e.g., over a threshold), the data may provide a recommendation (or dissuasion) of similar content or portions of the content to the user or to help the content developer improve the content for future users. For example, maybe the goal of the content developer is to increase stress in a video game, decrease stress for a meditation experience, or increase stress if a user is “bored” while studying or at work (e.g., to improve attention/respiration performance levels).

In some implementations, the method 700 or 800 further includes adjusting content corresponding to the experience based on the respiratory state and the attention state (e.g., customized to the respiratory state and the attention state of the user). For example, content recommendation for a content developer can be provided based on determining respiratory state and the attention states during the presented experience and changes of the experience or content presented therein. For example, the user may focus and meditate well when particular types of content are provided. In some implementations, the method 700 or 800 may further include identifying content based on similarity of the content to the experience, and providing a recommendation of the content to the user based on determining that the user has the respiratory state and attention state during the experience (e.g., mind wandering). In some implementations, the method 700 or 800 may further include customizing content included in the experience based on the respiratory state and the attention state of the user (e.g., breaking the content into smaller pieces).

In some implementations, content for the experience can be adjusted corresponding to the experience based on the respiratory state and attention state differing from an intended respiratory state and attention state for the experience. For example, content may be adjusted by an experience developer to improve recorded content for a subsequent use for the user or other users. In some implementations, the method 700 or 800 may further include adjusting content corresponding to the experience in response to determining that the respiratory state and attention state differs from a second respiratory state and attention state intended for the experience.

In some implementations, the method 700 or 800 may determine a context of the experience based on sensor data of the environment. For example, determining a context may involve using computer vision to generate a scene understanding of the visual and/or auditory attributes of the environment—where is the user, what is the user doing, what objects are nearby. Additionally, a scene understanding of the content presented to the user could be generated that includes the visual and/or auditory attributes of what the user was watching.

In some aspects, different contexts of the content presented and the environment are analyzed to determine where the user is, what the user is doing, what objects or people are nearby in the environment or within the content, what the user did earlier (e.g., meditated in the morning). Additionally, context analysis may include image analysis (semantic segmentation), audio analysis (jarring sounds), location sensors (where user is), motion sensors (fast moving vehicle), and even access other user data (e.g., a user's calendar). In an exemplary implementation, the method 700 or 800 may further include determining the context of the experience by generating a scene understanding of the environment based on the sensor data of the environment, the scene understanding including visual or auditory attributes of the environment and determining the context of the experience based on the scene understanding of the environment.

In some implementations, the sensor data includes image data, and generating the scene understanding is based at least on performing semantic segmentation of the image data and detecting one or more objects within the environment based on the semantic segmentation. In some implementations, determining the context of the experience includes determining an activity of the user based on the scene understanding of the environment. In some implementations, the sensor data includes location data of the user, and determining the context of the experience includes determining a location of the user within the environment based on the location data.

In some implementations, determining the context of the experience may involve identifying an object or individual with which the user is interacting. Determining the context of the experience may involve determining that the user is conversing with another individual. Determining the context of the experience may involve determining that an interaction or conversation with another individual is likely (or unlikely) to evoke a stressful state in the user. Assessing whether an individual is more or less likely to evoke a stressful response for the user may involve identifying the individual, and classifying the individual based on appearance of the individual, based on an action of the individual, and/or based on an activity that the individual is engaged in. For example, if the other individual is identified as the user's boss at work, the boss can be identified via facial recognition, or be classified as a supervising coworker. The stress of the user can then be tracked based on his or her respiratory state and the attention states when interacting with that individual that was classified as his or her boss. Providing feedback to a user (or his or her instructor) regarding the user's higher respiratory state and the attention states when interacting with his or her boss, may be useful when evaluating stress therapy techniques to better cope with high stress situations. Additionally, the shared experience may include a group of users sharing the common interest of meditating as a group, where the XR environment would enhance to the group's collective experience and/or an individual's experience.

In some implementations, determining the context of the experience may involve determining a scene understanding or scene knowledge that a particular location (e.g., a particular room, building, etc.) that a user experiences is more or less likely to lead to a stressful state (e.g., based on past stressful experiences that occurred there). Determining a scene understanding or scene knowledge of an experience may involve monitoring low level characteristics of a scene that can evoke stress. For example, loud noises, looming sounds, bright flashes of light, sirens, rumbling sounds, and the like, may be monitored and analyzed as part of the scene understanding or scene knowledge. Additionally, scene knowledge may provide information that a particular activity or content might be troubling or stressful. For example, scene knowledge may include experiences or event that the user is currently participating in such as interviewing, reading a disturbing news story, watching a scary movie, playing a violent video game, and the like. Understanding scene knowledge may involve other stressful experiences such as threatening stimuli (e.g., an aggressive dog), harm done to a loved one, perceived physical danger to the user (e.g., an oncoming car), online bullying, being berated in person, and the like.

In some implementations, determining the context of the experience includes determining an activity of the user based on a user's schedule. For example, the system may access a user's calendar to determine if a particular event is occurring when the particular respiratory state and the attention state is assessed (e.g., a scheduled meditation session, the user is late for an important meeting or class, or is scheduled to present in front of a group in the near future).

In some implementations, the techniques described herein obtain physiological data (e.g., pupillary data 40, EEG amplitude/frequency data, pupil modulation, eye gaze saccades, heart rate data, EDA data, etc.) from the user based on identifying typical interactions of the user with the experience. For example, the techniques may determine that a variability of an eye gaze characteristic of the user correlates with an interaction with the experience. Additionally, the techniques described herein may then adjust a visual characteristic of the experience or adjust/change a sound associated with the content enhancement, to enhance physiological response data associated with future interactions with the experience and/or the content enhancement presented within the experience. Moreover, in some implementations, changing a content enhancement after the user interacts with the experience informs the physiological response of the user in subsequent interactions with the experience or a particular segment of the experience. For example, the user may present an anticipatory physiological response associated with the change within the experience. Thus, in some implementations, the technique identifies an intent of the user to interact with the experience based on an anticipatory physiological response. For example, the technique may adapt or train an instruction set by capturing or storing physiological data of the user based on the interaction of the user with the experience, and may detect a future intention of the user to interact with the experience by identifying a physiological response of the user in anticipation of the presentation of the enhanced/updated experience.

In some implementations, an estimator or statistical learning method is used to better understand or make predictions about the physiological data (e.g., pupillary data characteristics, EEG data, EDA data, heart rate data, etc.). For example, statistics for EEG data may be estimated by sampling a dataset with replacement data (e.g., a bootstrap method).

In some implementations, the techniques could be trained on many sets of user physiological data and then adapted to each user individually. For example, content creators can customize an education experience (e.g., an instructional cooking video) based on the user physiological data, such as a user may require background music, different ambient lighting for learning, or require more or less audio or visual cues to continue to maintain meditation.

In some implementations, customization of the experience could be controlled by the user. For example, a user could select the experience he or she desires, such as he or she can choose the ambience, background scene, music, etc. Additionally, the user could alter the threshold of providing the content enhancement. For example, the user can customize the sensitivity of triggering the content enhancement based on prior experience of a session. For example, a user may desire to not have as many feedback notifications and allow some mind wandering (e.g., eye position deviations) before a notification is triggered. Thus, particular experiences can be customized on triggering a threshold when higher criteria is met. For example, some experiences, such as an education experience, a user may not want to be bothered during a study session if he or she is briefly staring off task or mind wandering by briefly looking towards a different area for a moment (e.g., less than 30 seconds) to contemplate what he or she just read. However, the student/reader would want to be given a notification if he or she is mind wandering for a longer period (e.g., longer than or equal to 30 seconds) by providing a content enhancement such as an auditory notification (e.g., “wake up”).

In some implementations, the techniques described herein can account for real-world environment 5 of the user 25 (e.g., visual qualities such as luminance, contrast, semantic context) in its evaluation of how much to modulate or adjust the presented content or content enhancements to enhance the physiological response (e.g., pupillary response) of the user 25 to the visual characteristic 30 (e.g., content enhancement).

In some implementations, the physiological data (e.g., pupillary data 40) may vary in time and the techniques described herein may use the physiological data to detect a pattern. In some implementations, the pattern is a change in physiological data from one time to another time, and, in some other implementations, the pattern is series of changes in physiological data over a period of time. Based on detecting the pattern, the techniques described herein can identify a change in the respiratory state and the attention state of the user and can then provide a content enhancement (e.g., visual or auditory cue to focus on breathing) to the user 25 to return to an intended state (e.g., lower respiratory state and the attention states) during an experience. For example, a respiratory state and the attention state of a user 25 may be identified by detecting a pattern in a user's gaze characteristic, heart rate, and/or PDA data, a visual or auditory cue associated with the experience may be adjusted (e.g., a content enhancement of a voice that states “focus on breathing” may further include a visual cue or a change in ambience of the scene), and the user's gaze characteristic, heart rate, and/or PDA data compared to the adjusted experience can be used to confirm the respiratory state and the attention state of a user.

In some implementations, the techniques described herein can utilize a training or calibration sequence to adapt to the specific physiological characteristics of a particular user 25. In some implementations, the techniques present the user 25 with a training scenario in which the user 25 is instructed to interact with on-screen items (e.g., feedback objects). By providing the user 25 with a known intent or area of interest (e.g., via instructions), the techniques can record the user's physiological data (e.g., pupillary data 40) and identify a pattern associated with the user's physiological data. In some implementations, the techniques can change a visual characteristic 30 (e.g., a content enhancement) associated with content 20 in order to further adapt to the unique physiological characteristics of the user 25. For example, the techniques can direct a user to mentally select a button associated with an identified area in the center of the screen on the count of three and record the user's physiological data (e.g., pupillary data 40) to identify a pattern associated with the user's respiratory state and the attention state. Moreover, the techniques can change or alter a visual characteristic associated with the content enhancement in order to identify a pattern associated with the user's physiological response to the altered visual characteristic. In some implementations, the pattern associated with the physiological response of the user 25 is stored in a user profile associated with the user and the user profile can be updated or recalibrated at any time in the future. For example, the user profile could automatically be modified over time during a user experience to provide a more personalized user experience (e.g., a personal educational experience for optimal learning experience while studying).

In some implementations, a machine learning model (e.g., a trained neural network) is applied to identify patterns in physiological data, including identification of physiological responses to presentation of content (e.g., content 20 of FIG. 1) during a particular experience (e.g., education, meditation, instructional, etc.). Moreover, the machine learning model may be used to match the patterns with learned patterns corresponding to indications of interest or intent of the user 25 to interact with the experience. In some implementations, the techniques described herein may learn patterns specific to the particular user 25. For example, the techniques may learn from determining that a peak pattern represents an indication of interest or intent of the user 25 in response to a particular visual characteristic 30 within the content and use this information to subsequently identify a similar peak pattern as another indication of interest or intent of the user 25. Such learning can take into account the user's relative interactions with multiple visual characteristics 30, in order to further adjust the visual characteristic 30 and enhance the user's physiological response to the experience and the presented content (e.g., focusing on particular areas of content versus other distracting areas).

In some implementations, the location and features of the head 27 of the user 25 (e.g., an edge of the eye, a nose or a nostril) are extracted by the device 10 and used in finding coarse location coordinates of the eyes 45 of the user 25, thus simplifying the determination of precise eye 45 features (e.g., position, gaze direction, etc.) and making the gaze characteristic(s) measurement more reliable and robust. Furthermore, the device 10 may readily combine the 3D location of parts of the head 27 with gaze angle information obtained via eye part image analysis in order to identify a given on-screen object at which the user 25 is looking at any given time. In some implementations, the use of 3D mapping in conjunction with gaze tracking allows the user 25 to move his or her head 27 and eyes 45 freely while reducing or eliminating the need to actively track the head 27 using sensors or emitters on the head 27.

By tracking the eyes 45, some implementations reduce the need to re-calibrate the user 25 after the user 25 moves his or her head 27. In some implementations, the device 10 uses depth information to track the pupil's 50 movement, thereby enabling a reliable present pupil diameter 55 to be calculated based on a single calibration of user 25. Utilizing techniques such as pupil-center-corneal reflection (PCCR), pupil tracking, and pupil shape, the device 10 may calculate the pupil diameter 55, as well as a gaze angle of the eye 45 from a fixed point of the head 27, and use the location information of the head 27 in order to re-calculate the gaze angle and other gaze characteristic(s) measurements. In addition to reduced recalibrations, further benefits of tracking the head 27 may include reducing the number of light projecting sources and reducing the number of cameras used to track the eye 45.

In some implementations, the techniques described herein can identify a particular object within the content presented on the display 15 of the device 10 at a position in the direction of the user's gaze. Moreover, the techniques can change a state of the visual characteristic 30 associated with the particular object or the overall content experience responsively to a spoken verbal command received from the user 25 in combination with the identified respiratory state and the attention state of the user 25. For example, a particular object within the content may be an icon associated with a software application, and the user 25 may gaze at the icon, say the word “select” to choose the application, and a highlighting effect may be applied to the icon. The techniques can then use further physiological data (e.g., pupillary data 40) in response to the visual characteristic 30 (e.g., a content enhancement) to further identify a respiratory state and the attention state of the user 25 as a confirmation of the user's verbal command. In some implementations, the techniques can identify a given interactive item responsive to the direction of the user's gaze, and to manipulate the given interactive item responsively to physiological data (e.g., variability of the gaze characteristics). The techniques can then confirm the direction of the user's gaze based on further identifying respiratory state and the attention states of a user with physiological data in response to interactions with the experience (e.g., interacting within an intense video game). In some implementations, the techniques can remove an interactive item or object based on the identified interest or intent. In other implementations, the techniques can automatically capture images of the content at times when an interest or intent of the user 25 is determined.

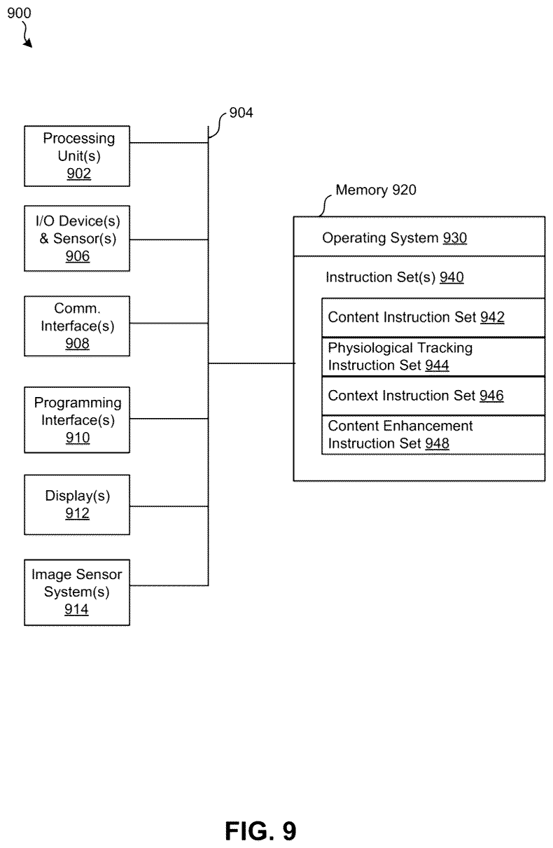

FIG. 9 is a block diagram of an example device 900. Device 900 illustrates an exemplary device configuration for device 10. While certain specific features are illustrated, those skilled in the art will appreciate from the present disclosure that various other features have not been illustrated for the sake of brevity, and so as not to obscure more pertinent aspects of the implementations disclosed herein. To that end, as a non-limiting example, in some implementations the device 10 includes one or more processing units 902 (e.g., microprocessors, ASICs, FPGAs, GPUs, CPUs, processing cores, and/or the like), one or more input/output (I/O) devices and sensors 906, one or more communication interfaces 908 (e.g., USB, FIREWIRE, THUNDERBOLT, IEEE 802.3x, IEEE 802.11x, IEEE 802.16x, GSM, CDMA, TDMA, GPS, IR, BLUETOOTH, ZIGBEE, SPI, 12C, and/or the like type interface), one or more programming (e.g., I/O) interfaces 910, one or more displays 912, one or more interior and/or exterior facing image sensor systems 914, a memory 920, and one or more communication buses 904 for interconnecting these and various other components.

In some implementations, the one or more communication buses 904 include circuitry that interconnects and controls communications between system components. In some implementations, the one or more I/O devices and sensors 906 include at least one of an inertial measurement unit (IMU), an accelerometer, a magnetometer, a gyroscope, a thermometer, one or more sensors that obtain physiological data (e.g., blood pressure monitor, heart rate monitor, blood oxygen sensor, blood glucose sensor, etc.), one or more microphones, one or more speakers, a haptics engine, one or more depth sensors (e.g., a structured light, a time-of-flight, or the like), and/or the like.

In some implementations, the one or more displays 912 are configured to present a view of a physical environment or a graphical environment to the user. In some implementations, the one or more displays 912 correspond to holographic, digital light processing (DLP), liquid-crystal display (LCD), liquid-crystal on silicon (LCoS), organic light-emitting field-effect transitory (OLET), organic light-emitting diode (OLED), surface-conduction electron-emitter display (SED), field-emission display (FED), quantum-dot light-emitting diode (QI-LED), micro-electro-mechanical system (MEMS), and/or the like display types. In some implementations, the one or more displays 912 correspond to diffractive, reflective, polarized, holographic, etc. waveguide displays. In one example, the device 10 includes a single display. In another example, the device 10 includes a display for each eye of the user.

In some implementations, the one or more image sensor systems 914 are configured to obtain image data that corresponds to at least a portion of the physical environment 5. For example, the one or more image sensor systems 914 include one or more RGB cameras (e.g., with a complimentary metal-oxide-semiconductor (CMOS) image sensor or a charge-coupled device (CCD) image sensor), monochrome cameras, IR cameras, depth cameras, event-based cameras, and/or the like. In various implementations, the one or more image sensor systems 914 further include illumination sources that emit light, such as a flash. In various implementations, the one or more image sensor systems 914 further include an on-camera image signal processor (ISP) configured to execute a plurality of processing operations on the image data.

The memory 920 includes high-speed random-access memory, such as DRAM, SRAM, DDR RAM, or other random-access solid-state memory devices. In some implementations, the memory 920 includes non-volatile memory, such as one or more magnetic disk storage devices, optical disk storage devices, flash memory devices, or other non-volatile solid-state storage devices. The memory 920 optionally includes one or more storage devices remotely located from the one or more processing units 902. The memory 920 includes a non-transitory computer readable storage medium.

In some implementations, the memory 920 or the non-transitory computer readable storage medium of the memory 920 stores an optional operating system 930 and one or more instruction set(s) 940. The operating system 930 includes procedures for handling various basic system services and for performing hardware dependent tasks. In some implementations, the instruction set(s) 940 include executable software defined by binary information stored in the form of electrical charge. In some implementations, the instruction set(s) 940 are software that is executable by the one or more processing units 902 to carry out one or more of the techniques described herein.

The instruction set(s) 940 include a content instruction set 942, a physiological tracking instruction set 944, a context instruction set 946, and a respiratory state and the contact enhancement instruction set 948. The instruction set(s) 940 may be embodied a single software executable or multiple software executables.

In some implementations, the content instruction set 942 is executable by the processing unit(s) 902 to provide and/or track content for display on a device. The content instruction set 942 may be configured to monitor and track the content over time (e.g., during an experience such as an education session) and/or to identify change events that occur within the content. In some implementations, the content instruction set 942 may be configured to inject change events into content (e.g., content enhancements) using one or more of the techniques discussed herein or as otherwise may be appropriate. To these ends, in various implementations, the instruction includes instructions and/or logic therefor, and heuristics and metadata therefor.

In some implementations, the physiological tracking instruction set 944 is executable by the processing unit(s) 902 to track a user's physiological attributes (e.g., EEG amplitude/frequency, pupil modulation, eye gaze saccades, heart rate, EDA data, etc.) using one or more of the techniques discussed herein or as otherwise may be appropriate. To these ends, in various implementations, the instruction includes instructions and/or logic therefor, and heuristics and metadata therefor.

In some implementations, the context instruction set 946 is executable by the processing unit(s) 902 to determine a context of the experience and/or the environment (e.g., create a scene understanding to determine the objects or people in the content or in the environment, where the user is, what the user is watching, etc.) using one or more of the techniques discussed herein (e.g., object detection, facial recognition, etc.) or as otherwise may be appropriate. To these ends, in various implementations, the instruction includes instructions and/or logic therefor, and heuristics and metadata therefor.

In some implementations, the respiratory state and the contact enhancement instruction set 948 is executable by the processing unit(s) 902 to assess the respiratory state and the attention state (e.g., focused, distracted, etc.) of a user based on physiological data (e.g., eye gaze response) and context data of the content and/or environment using one or more of the techniques discussed herein or as otherwise may be appropriate. To these ends, in various implementations, the instruction includes instructions and/or logic therefor, and heuristics and metadata therefor.

Although the instruction set(s) 940 are shown as residing on a single device, it should be understood that in other implementations, any combination of the elements may be located in separate computing devices. Moreover, FIG. 9 is intended more as functional description of the various features which are present in a particular implementation as opposed to a structural schematic of the implementations described herein. As recognized by those of ordinary skill in the art, items shown separately could be combined and some items could be separated. The actual number of instructions sets and how features are allocated among them may vary from one implementation to another and may depend in part on the particular combination of hardware, software, and/or firmware chosen for a particular implementation.

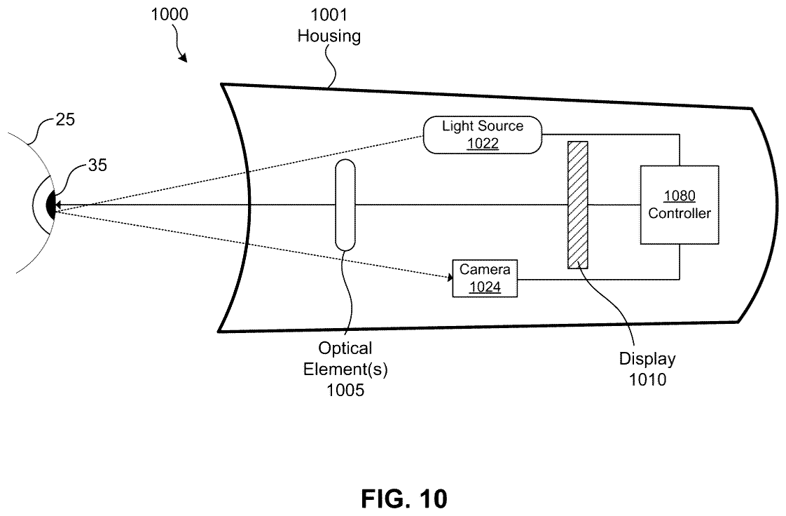

FIG. 10 illustrates a block diagram of an exemplary head-mounted device 1000 in accordance with some implementations. The head-mounted device 1000 includes a housing 1001 (or enclosure) that houses various components of the head-mounted device 1000. The housing 1001 includes (or is coupled to) an eye pad (not shown) disposed at a proximal (to the user 25) end of the housing 1001. In various implementations, the eye pad is a plastic or rubber piece that comfortably and snugly keeps the head-mounted device 1000 in the proper position on the face of the user 25 (e.g., surrounding the eye of the user 25).

The housing 1001 houses a display 1010 that displays an image, emitting light towards or onto the eye of a user 25. In various implementations, the display 1010 emits the light through an eyepiece having one or more optical elements 1005 that refracts the light emitted by the display 1010, making the display appear to the user 25 to be at a virtual distance farther than the actual distance from the eye to the display 1010. For example, optical element(s) 1005 may include one or more lenses, a waveguide, other diffraction optical elements (DOE), and the like. For the user 25 to be able to focus on the display 1010, in various implementations, the virtual distance is at least greater than a minimum focal distance of the eye (e.g., 6 cm). Further, in order to provide a better user experience, in various implementations, the virtual distance is greater than 1 meter.

The housing 1001 also houses a tracking system including one or more light sources 1022, camera 1024, and a controller 1080. The one or more light sources 1022 emit light onto the eye of the user 25 that reflects as a light pattern (e.g., a circle of glints) that can be detected by the camera 1024. Based on the light pattern, the controller 1080 can determine an eye tracking characteristic of the user 25. For example, the controller 1080 can determine a gaze direction and/or a blinking state (eyes open or eyes closed) of the user 25. As another example, the controller 1080 can determine a pupil center, a pupil size, or a point of regard. Thus, in various implementations, the light is emitted by the one or more light sources 1022, reflects off the eye of the user 25, and is detected by the camera 1024. In various implementations, the light from the eye of the user 25 is reflected off a hot mirror or passed through an eyepiece before reaching the camera 1024.

The display 1010 emits light in a first wavelength range and the one or more light sources 1022 emit light in a second wavelength range. Similarly, the camera 1024 detects light in the second wavelength range. In various implementations, the first wavelength range is a visible wavelength range (e.g., a wavelength range within the visible spectrum of approximately 400-800 nm) and the second wavelength range is a near-infrared wavelength range (e.g., a wavelength range within the near-infrared spectrum of approximately 700-1400 nm).

In various implementations, eye tracking (or, in particular, a determined gaze direction) is used to enable user interaction (e.g., the user 25 selects an option on the display 1010 by looking at it), provide foveated rendering (e.g., present a higher resolution in an area of the display 1010 the user 25 is looking at and a lower resolution elsewhere on the display 1010), or correct distortions (e.g., for images to be provided on the display 1010).

In various implementations, the one or more light sources 1022 emit light towards the eye of the user 25 which reflects in the form of a plurality of glints.

In various implementations, the camera 1024 is a frame/shutter-based camera that, at a particular point in time or multiple points in time at a frame rate, generates an image of the eye of the user 25. Each image includes a matrix of pixel values corresponding to pixels of the image which correspond to locations of a matrix of light sensors of the camera. In implementations, each image is used to measure or track pupil dilation by measuring a change of the pixel intensities associated with one or both of a user's pupils.

In various implementations, the camera 1024 is an event camera including a plurality of light sensors (e.g., a matrix of light sensors) at a plurality of respective locations that, in response to a particular light sensor detecting a change in intensity of light, generates an event message indicating a particular location of the particular light sensor.

It will be appreciated that the implementations described above are cited by way of example, and that the present invention is not limited to what has been particularly shown and described hereinabove. Rather, the scope includes both combinations and sub combinations of the various features described hereinabove, as well as variations and modifications thereof which would occur to persons skilled in the art upon reading the foregoing description and which are not disclosed in the prior art.

As described above, one aspect of the present technology is the gathering and use of physiological data to improve a user's experience of an electronic device with respect to interacting with electronic content. The present disclosure contemplates that in some instances, this gathered data may include personal information data that uniquely identifies a specific person or can be used to identify interests, traits, or tendencies of a specific person. Such personal information data can include physiological data, demographic data, location-based data, telephone numbers, email addresses, home addresses, device characteristics of personal devices, or any other personal information.

The present disclosure recognizes that the use of such personal information data, in the present technology, can be used to the benefit of users. For example, the personal information data can be used to improve interaction and control capabilities of an electronic device. Accordingly, use of such personal information data enables calculated control of the electronic device. Further, other uses for personal information data that benefit the user are also contemplated by the present disclosure.

The present disclosure further contemplates that the entities responsible for the collection, analysis, disclosure, transfer, storage, or other use of such personal information and/or physiological data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

Despite the foregoing, the present disclosure also contemplates implementations in which users selectively block the use of, or access to, personal information data. That is, the present disclosure contemplates that hardware or software elements can be provided to prevent or block access to such personal information data. For example, in the case of user-tailored content delivery services, the present technology can be configured to allow users to select to “opt in” or “opt out” of participation in the collection of personal information data during registration for services. In another example, users can select not to provide personal information data for targeted content delivery services. In yet another example, users can select to not provide personal information, but permit the transfer of anonymous information for the purpose of improving the functioning of the device.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences or settings based on non-personal information data or a bare minimum amount of personal information, such as the content being requested by the device associated with a user, other non-personal information available to the content delivery services, or publicly available information.

In some embodiments, data is stored using a public/private key system that only allows the owner of the data to decrypt the stored data. In some other implementations, the data may be stored anonymously (e.g., without identifying and/or personal information about the user, such as a legal name, username, time and location data, or the like). In this way, other users, hackers, or third parties cannot determine the identity of the user associated with the stored data. In some implementations, a user may access his or her stored data from a user device that is different than the one used to upload the stored data. In these instances, the user may be required to provide login credentials to access their stored data.

In general, one innovative exemplary embodiment of the subject matter described in this specification can be embodied in methods at a device including a processor that include the actions of presenting content to a user and obtaining physiological data associated with the user via one or more sensors, determining a respiratory state of the user based on the physiological data, determining an attention state of the user based on the physiological data, determining a modification to the content based on the respiratory state and the attention state of the user, and presenting the modified content to the user.

These and other exemplary embodiments can each optionally include one or more of the following features.

In some embodiments, the method further includes presenting an instruction for the user to be attentive to breathing, and assessing a level of attentiveness to breathing based on the respiratory state and attention state, where the modification is determined based on the level of attentiveness.

In some embodiments, determining the modification includes determining a baseline corresponding to the user based on the respiratory state and attention state, determining a goal for the user based on the baseline, and determining the modification based on the baseline and the goal.

In some embodiments, the modification includes initiation of a new meditation, conclusion of a meditation, or changing of an ongoing meditation. In some embodiments, the modification includes a visual or audible representation of the respiratory state or a change to the respiratory state. In some exemplary embodiments, the modification includes a visual or audible representation of the respiratory state; and a visual or audible representation of the attention state. In some exemplary embodiments, the modification includes a cue configured to trigger a change in the respiratory state or attention state. In some embodiments, the modification includes a graphical indicator or sound configured to change the attention state to a different attention state.

In some embodiments, the modification includes a visual or audible indication of a suggested time for an experience.

In some embodiments, the respiratory state is determined based on using the physiological data to determine head pose, sounds, jaw movement, cheek movement, nose movement, movement of tissue surrounding an eye, or a signal of a face modulated by breath.

In some embodiments, the attention state is determined based on using the physiological data to measure gaze or body stability. In some aspects, the attention state is determined based on determining a level of attentiveness. In some aspects, the attention state is determined based on one or more physiological data streams via an output of the one or more sensors.

In some embodiments, the method further includes identifying emotional states of the user corresponding to multiple periods of time and presenting indications of progress based on the emotional states.

In some embodiments, the content includes an extended reality (XR) experience. In some embodiments, the content is presented to multiple users during a communication session. In some embodiments, the device is or includes a head-mounted device (HMD).

Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter.

Unless specifically stated otherwise, it is appreciated that throughout this specification discussions utilizing the terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general purpose computing apparatus to a specialized computing apparatus implementing one or more implementations of the present subject matter. Any suitable programming, scripting, or other type of language or combinations of languages may be used to implement the teachings contained herein in software to be used in programming or configuring a computing device.

Implementations of the methods disclosed herein may be performed in the operation of such computing devices. The order of the blocks presented in the examples above can be varied for example, blocks can be re-ordered, combined, or broken into sub-blocks. Certain blocks or processes can be performed in parallel.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various objects, these objects should not be limited by these terms. These terms are only used to distinguish one object from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “or” as used herein refers to and encompasses any and all possible combinations of one or more of the associated listed items. It will be further understood that the terms “comprises” or “comprising,” when used in this specification, specify the presence of stated features, integers, steps, operations, objects, or components, but do not preclude the presence or addition of one or more other features, integers, steps, operations, objects, components, or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The foregoing description and summary of the invention are to be understood as being in every respect illustrative and exemplary, but not restrictive, and the scope of the invention disclosed herein is not to be determined only from the detailed description of illustrative implementations but according to the full breadth permitted by patent laws. It is to be understood that the implementations shown and described herein are only illustrative of the principles of the present invention and that various modification may be implemented by those skilled in the art without departing from the scope and spirit of the invention.

1-55. (canceled)

56. A method comprising:
at a device comprising a processor:
obtaining physiological data via one or more sensors;
determining an attentive state during a meditation mode based on the physiological data;
customizing feedback content for guiding a change of the attentive state during the meditation mode, wherein the feedback content is customized based on a user attribute; and
providing, after a delay time based on the user attribute, the customized feedback content during the meditation mode.

57. The method of claim 56, further comprising:
in response to providing the customized feedback content, determining a level of attentiveness based on the physiological data for a period of time; and
determining a feedback metric based on the determined level of attentiveness for the period of time.

58. The method of claim 56, wherein the user attribute comprises a meditation experience level.

59. The method of claim 58, wherein the meditation experience level is determined based on accessing a user profile, an analysis of prior meditation experiences, or a combination thereof.

60. The method of claim 58, wherein the meditation experience level is updated based on the attentive state during the meditation mode.

61. The method of claim 56, further comprising:
presenting an instruction to be attentive to breathing; and
assessing a level of attentiveness to breathing based on a respiratory state and the attentive state, wherein the customized feedback is determined based on the level of attentiveness.

62. The method of claim 56, wherein customizing the feedback content comprises:
determining a baseline based on the physiological data;
determining a goal based on the baseline; and
determining the customized feedback content based on the baseline and the goal.

63. The method of claim 56, further comprising:
identifying meditation states corresponding to multiple periods of time; and
presenting indications of progress based on the meditation states.

64. The method of claim 56, further comprising:
determining a context of the meditation mode by generating a scene understanding of an environment of the meditation mode based on sensor data from the one or more sensors, the scene understanding comprising visual or auditory attributes of the environment; and
customizing the feedback content based on the context of the meditation mode.

65. The method of claim 56, wherein the customized feedback content comprises:
conclusion of a current meditation mode;
initiation of another meditation mode different than the current meditation mode; or
changing of the current meditation mode.

66. The method of claim 56, wherein the customized feedback content comprises a volume of an audio signal that is modulated based on the physiological data.

67. The method of claim 56, wherein the customized feedback content comprises a visual or audible representation of the attentive state or a change to the attentive state.

68. The method of claim 56, wherein the customized feedback content comprises a cue configured to trigger a change in the attentive state.

69. The method of claim 56, wherein the customized feedback content comprises a graphical indicator or sound configured to change a first attentive state to a second attentive state.

70. The method of claim 56, wherein the customized feedback content comprises a visual or audible indication of a suggested time for a new meditation experience.

71. The method of claim 56, wherein the attentive state is determined based on using the physiological data to measure gaze or body stability, determining a level of attentiveness, determining a level of a respiratory state, or a combination thereof.

72. The method of claim 56, wherein the physiological data includes at least one of skin temperature, respiration, photoplethysmogram (PPG), electrodermal activity (EDA), eye gaze tracking, and pupillary movement.

73. The method of claim 56, wherein the meditation mode is presented in multiple instances during a communication session.

74. A device comprising:
a non-transitory computer-readable storage medium; and
one or more processors coupled to the non-transitory computer-readable storage medium, wherein the non-transitory computer-readable storage medium comprises program instructions that, when executed on the one or more processors, cause the one or more processors to perform operations comprising:
obtaining physiological data via one or more sensors;
determining an attentive state during a meditation mode based on the physiological data;
customizing feedback content for guiding a change of the attentive state during the meditation mode, wherein the feedback content is customized based on a user attribute; and
providing, after a delay time based on the user attribute, the customized feedback content during the meditation mode.

75. A non-transitory computer-readable storage medium, storing program instructions executable on a device to perform operations comprising:
obtaining physiological data via one or more sensors;
determining an attentive state during a meditation mode based on the physiological data;
customizing feedback content for guiding a change of the attentive state during the meditation mode, wherein the feedback content is customized based on a user attribute; and
providing, after a delay time based on the user attribute, the customized feedback content during the meditation mode.
